(19) United States
(12) Patent Application Publication
(10) Pub. No.: US 2026/0347380 A1
(43) Pub. Date: Nov. 5, 2026

(54) MODULATORS OF CD1 PROTEIN BINDING TO T CELL RECEPTORS

(71) Applicants: The Brigham and Women's Hospital, Inc., Boston, MA (US); Monash University, Clayton (AU); University of Oxford, Oxford (GB); The University of Birmingham, Edgbaston (GB)

(72) Inventors: David Branch Moody, Boston, MA (US); Tan-Yun Cheng, Boston, MA (US); Jamie Rossjohn, Carnegie (AU); Graham S. Ogg, Oxfordshire (GB); Clare Susan Suckling, Oxfordshire (GB); Gurdyal S. Besra, West Midlands (GB)

(21) Appl. No.: 19/114,687

(22) PCT Filed: Sep. 29, 2023
(86) PCT No.: PCT/US2023/075526

§ 371 Date: Mar. 24, 2025

Related U.S. Application Data
(60) Provisional application No. 63/411,971, filed on Sep. 30, 2022.

(51) Int. Cl. C07F 9/113; A61K 9/00; A61K 31/688; A61K 31/7032; A61P 29/00; C07H 15/10

(52) U.S. Cl. CPC C07F 9/113; A61K 9/0014; A61K 31/688; A61K 31/7032; A61P 29/00; C07H 15/10

(57) ABSTRACT

The present disclosure provides lipid compounds useful in treating inflammatory skin diseases.

### CLAIM OF PRIORITY

This application claims priority U.S. Patent Application Ser. No. 63/411,971, filed on Sep. 30, 2022, the entire contents of which are hereby incorporated by reference.

### FEDERALLY SPONSORED RESEARCH OR DEVELOPMENT

This disclosure was made with Government support under Grant No. AR048632 and AI049313 awarded by the National Institutes of Health. The Government has certain rights in the invention.

### TECHNICAL FIELD

This disclosure relates to lipid compounds, and in particular to synthetic derivatives of sphingosine and unsaturated very long chain fatty acids (VLCFA) useful in treating inflammatory diseases, where CD1 proteins are implicated, such as inflammatory diseases of the skin.

### BACKGROUND

Inflammatory skin diseases present a significant socioeconomic burden. Acne, contact dermatitis, atopic dermatitis, and psoriasis remain highly prevalent skin diseases that are only partially treated by current therapies, including those with non-specific effects on the immune system and skin, creating unwanted side effects. Spending on dermatitis and acne therapies exceeds 28 billion dollars per year worldwide and is constantly increasing. In a similar manner, psoriasis is a chronic, immune-mediated, inflammatory skin disease with a profound impact on patients' quality of life. Psoriasis and acne may lead to social isolation, anxiety, distress and reduced self-esteem. Psoriasis patients experience a range of symptoms including itching and plaque-related pain. They are also at an increased risk of developing comorbidities, including depression, psoriatic arthritis, inflammatory bowel disease, metabolic disease, cardiovascular disease and diabetes. The impact of psoriasis and other inflammatory skin disease is not only limited to those suffering from the disease, but also represents a significant burden on the health care system. Psoriasis, atopic dermatitis, and other inflammatory skin diseases lead to increased healthcare spending and limits employment opportunities and income of those affected by these conditions and their co-morbidities.

CD1-restricted T cells have been implicated in inflammatory conditions such as psoriasis, atopic dermatitis, and contact dermatitis. CD1-restricted T cells recognize lipid antigens and can be divided into 2 groups, group 1 CD1 (CD1a, b, c)-restricted and group 2 CD1d-restricted T cells (NKT cells). Several findings have made them prime suspects in the pathogenesis of dermatological inflammatory conditions.

### SUMMARY

In some embodiments, the present disclosure provides a compound of Formula (I):

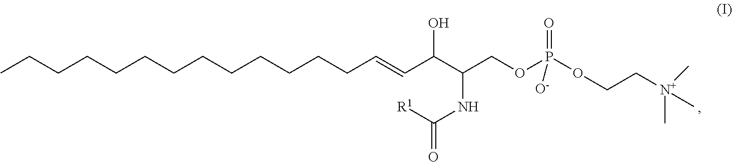

or a pharmaceutically acceptable salt thereof, wherein R¹ is as described herein.

In some embodiments, the present disclosure provides a compound of Formula (II):

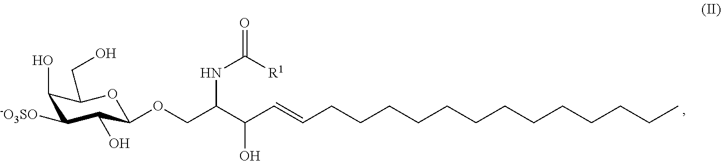

or a pharmaceutically acceptable salt thereof, wherein R¹ is as described herein.

In some embodiments, the present disclosure provides a pharmaceutical composition comprising a compound of Formula (I) or Formula (II), or a pharmaceutically acceptable salt thereof, and a pharmaceutically acceptable carrier. In some embodiments, the pharmaceutical composition is suitable for topical administration.

In some embodiments, the present disclosure provides a method of preventing or treating an inflammatory disease, the method comprising administering to a subject in need thereof a therapeutically effective amount of a compound of Formula (I) or Formula (II), or a pharmaceutically acceptable salt thereof, or a pharmaceutical composition comprising same. In some embodiments, the inflammatory disease is an inflammatory skin disease.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains a schematic showing T cell receptors binding peptide-MHC or lipid-CD1 complexes.

FIG. 2 contains a schematic showing natural lipids binding CD1a and blocking T cell response.

FIG. 3 shows that the long chain doubly unsaturated sphingomyelin (42:2 SM) binds CD1a with its phosphocholine headgroup protruding, which blocks approach of T cell receptors (42:2 SM). The more common short chain 36:2 SM binds by tucking its phosphocholine group largely inside CD1a so T cell receptors can approach CD1a and are not blocked.

FIG. 4 shows that sulfatide lipid compound with a green and yellow head group forms complex with CD1a. Sulfatide sits similarly within CD1a as compared to other lipids, but the sulfatide headgroup alters the protein surface of CD1a and protrudes to the outer surface of CD1a where it can block T cell receptors.

FIG. 5 shows inhibition of CD1a binding to human T cells in vitro. Exemplified sphingomyelins block CD1a protein binding to RCS36 T cells. Fluorescent human CD1a tetramers were treated with media (mock) or a sphingomyelin variant synthesized with the indicated fatty chain and tested for staining of human CD1a autoreactive T cells using flow cytometry. In all cases the fatty acyl group is conjugated to a C18:1 sphingosine unit. Thus, the C28:1 fatty acyl group and the C18:1 sphingosine group constitute C46:2 sphingomyelin (compound 10).

FIG. 6 shows sphingomyelin intervention model to modulate skin inflammation in vivo. Sphingomyelin species C34:1, C42:2 or C46:2 (20 μg/20 μl ethanol), or vehicle alone (20 μl ethanol) were applied topically to WT or CD1a transgenic mouse ear skin at 48 hour intervals before and during application of Aldara on days 0-5 (20 mg 5% imiquimod cream).

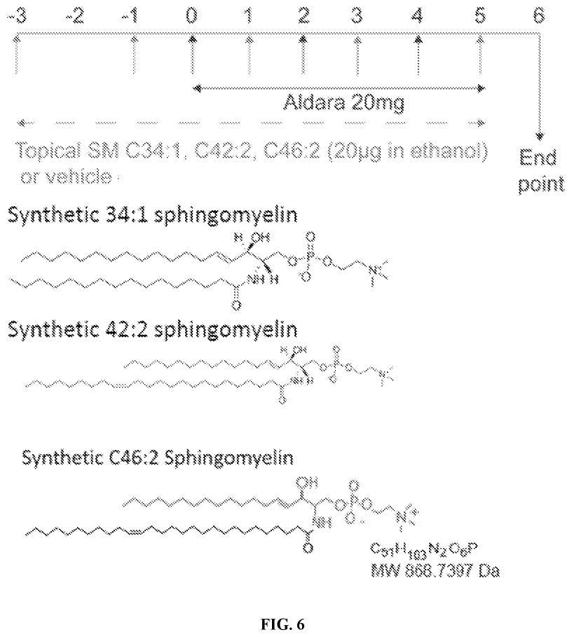

FIG. 7 shows that application of long chain sphingomyelins in vivo reduces imiquimod-induced skin thickening. Daily measurement of ear swelling induced by imiquimod treatment of wild-type (WT) and CD1a transgenic mice (CD1a) treated topically with 20 μg sphingomyelin (SM34:1, SM42:2 or SM46:2) dissolved in ethanol, or ethanol alone control, as in FIG. 6. (N=2-3, 2-way-ANOVA with Dunnett's test, *, P<0.05; ****, P<0.0001 indicates significance on comparison to “CD1a:EtOH” at day 7, mean±SD).

FIG. 8 shows that application of very long chain sphingomyelins in vivo reduces imiquimod-induced skin inflammation. Representative images of imiquimod-treated wild-type (WT) and CD1a transgenic mouse ear skin (CD1a) treated topically with 20 μg sphingomyelin (SM34:1, SM42:2 or SM46:2) dissolved in ethanol, or ethanol alone control, as in FIG. 6, images taken on day 7. Steroid is mometasone cream.

FIG. 9A shows the impact of sphingomyelin application on the imiquimod-induced cutaneous cellular immune response. Flow cytometry analysis of ear skin of imiquimod-treated wild-type (WT) and CD1a transgenic mice (CD1a) treated topically with 20 μg sphingomyelin (SM34:1, SM42:2 or SM46:2) dissolved in ethanol, or ethanol alone control, as in FIG. 6. Skin T cell and draining lymph node T cell, frequency was determined. And lymph node CD4 and CD8 T cells assessed for cell surface CD69 expression, which is an indication of T cell activation. Skin and draining LN eosinophil and neutrophil frequency was determined. (N=2-3, 1-way-ANOVA with Dunnett's test, *, P<0.05; **, P<0.01; ***, P<0.001; ****, P<0.0001, mean±SD).

FIG. 9B shows the impact of sphingomyelin application on the imiquimod-induced cutaneous cytokine-based immune response. Skin cytokine levels were assessed on day 7 of imiquimod-treated wild-type (WT) and CD1a transgenic mice (CD1a) treated topically with 20 μg sphingomyelin (SM34:1, SM42:2 or SM46:2) dissolved in ethanol, or ethanol alone control, as in FIG. 6, as measured by cytometric bead array. (N=2-3, 1-way-ANOVA with Dunnett's test, *, P<0.05; **, P<0.01; ***, P<0.001; ****, P<0.0001, mean±SD).

FIG. 10 shows the impact of sphingomyelin application on the imiquimod-induced systemic immune response. Plasma cytokine levels of the blood of imiquimod-treated wild-type (WT) and CD1a transgenic mice (CD1a) treated topically with 20 μg sphingomyelin (SM34:1, SM42:2 or SM46:2) dissolved in ethanol, or ethanol alone control, as in FIG. 6, as measured by cytometric bead array. (N=2-3, 1-way-ANOVA with Dunnett's test, *, P<0.05; **, P<0.01, mean±SD).

FIG. 11 show the impact of sphingomyelin application on MC903 type 2 inflammation and clinical itch. The ears of hCD1a and WT mice were topically treated with 1 nmol MC903/EtOH on days 0, 2 and 5. 20 μg/20 μl of SM 46:2, SM 42:2, SM 34:1 and vehicle were applied on the ear tissue on days-2, 0, 2 and 4. (A) Ear thickness, and (B) itching frequency were evaluated at endpoint of day 6. Each data plot represents an individual ear. Plots show mean±SEM. Statistics were calculated using one-way ANOVA with Tukey post-test. *** 0.01<P<0.001, **** P<0.001, NS=nonsignificant.

### DETAILED DESCRIPTION

Without being bound by any particular theory, it is considered that CD1 proteins are expressed locally in defined tissues, such as the skin, where they bind lipids that activate human T cells to cause inflammation. The present disclosure is based, at least in part, on the realization that synthetic lipids such as those within the present claims bind CD1 but block activation of T cells, which effectively treats skin inflammation.

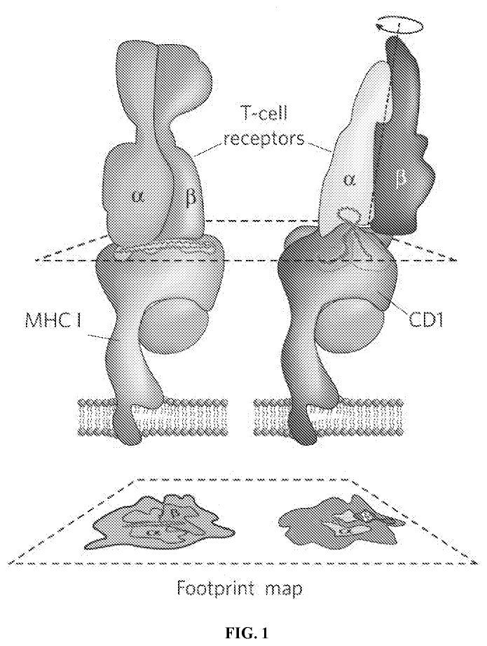

T cells recognize millions of antigens using rearranged and clonally distributed T cell receptors (TCRs). Activated T cells are the central effectors of many types of human immune response, as they orchestrate the actions of other T cells, B cells, dendritic cells and Natural Killer (NK) cells that are key causes of human autoimmune diseases. Many studies of T cell mediated autoimmunity emphasize that TCRs control T cell activation, which occurs in response to peptide antigens presented by highly polymorphic Major Histocompatibility Complex (MHC) proteins (See FIG. 1, left). Because MHC genes are polymorphic, MHC antigen presenting proteins differ in their structure from person to person. Therefore, engineering of broadly activating or suppressive ligands of MHC proteins has not been broadly feasible for application in human populations. In contrast, the genes encoding human CD1 antigen presenting molecules are encoded outside the MHC, and functionally significant polymorphisms are extremely rare. Thus, CD1 proteins expressed by genetically unrelated individuals have similar or identical antigen binding clefts. Therefore, the simplified population genetics of the CD1 system offers an opportunity to take advantage of CD1 ligands discovered in human systems and then design individual lipid ligands that can be applied to affect T cells from nearly any human donor.

A second major feature of CD1 proteins is that they bind lipids (See FIG. 1, right), whereas MHC I and II bind and display peptides. As contrasted with the many peptide antigen agonists and antagonists of T cell responses that have been extensively investigated, lipid ligands of CD1 proteins have not been as broadly described and implemented as modulators of T cell function. In particular, the full scope of lipids from human cells bound by CD1a, CD1b, CD1c and CD1d proteins has not been systematically reported. T cell antigen receptors provide strong, primary signals that control the activation, de-activation or death of T cells. Without being bound by any particular theory or speculation, it is considered that the lipidic compounds within the present claims modulate T cell autoimmunity by modulating T cell receptor contact with non-polymorphic CD1 proteins. Engineering lipid ligands that efficiently bind CD1 proteins and broadly block TCR binding and activation could selectively inhibit CD1-reactive T cells without substantially changing other aspects of the immune system.

The four types of human CD1 antigen presenting proteins, CD1a, CD1b, CD1c and CD1d, are expressed at high density on dendritic cells, macrophages, and B cells, which are the major antigen presenting cells in the human body. CD1 proteins bind many types of lipids to form complexes that engage T cell receptors (e.g., αβTCRs or γδTCRs) leading to T cell activation. CD1a, CD1b, CD1c and CD1d proteins each have a distinct lipid binding clefts that accommodate differing patterns of lipid ligands. Newly synthesized CD1 proteins fold in the endoplasmic reticulum, where they capture lipids and traffic as CD1-lipid complexes through the secretory pathway to the cell surface. A cohort of CD1 proteins on a given cell bind diverse lipids, which generates CD1 lipid complexes that either do or do not bind to the major activating receptors on T cells known as αβ T cell receptors or γδ T cell receptors. CD1 proteins can capture self or foreign lipids for display to T cells.

In the most studied examples, CD1 ligands use their aliphatic hydrocarbon tails, also known as lipid anchors, to bind within a hydrophobic cleft in CD1. This mode of binding allows the carbohydrate, sulfate, choline or other hydrophilic head groups to protrude to the surface of CD1 (See FIG. 1). The TCR can recognize an epitope that is formed by the surface of CD1 itself (i.e., CD1 autoreactive T cells) or by a combination of the CD1 protein and the protruding head group of each lipid antigen (lipid reactive T cell). Most previously studied lipid ligands of CD1 were discovered in activation assays and have activating properties for T cells. Without being bound to any particular theory, the CD1 ligands described in this application were discovered through binding assays. Therefore, the ligands reported have particularly strong CD1 binding properties, but can lead to activation, no effect, or blockade of functional T cell responses.

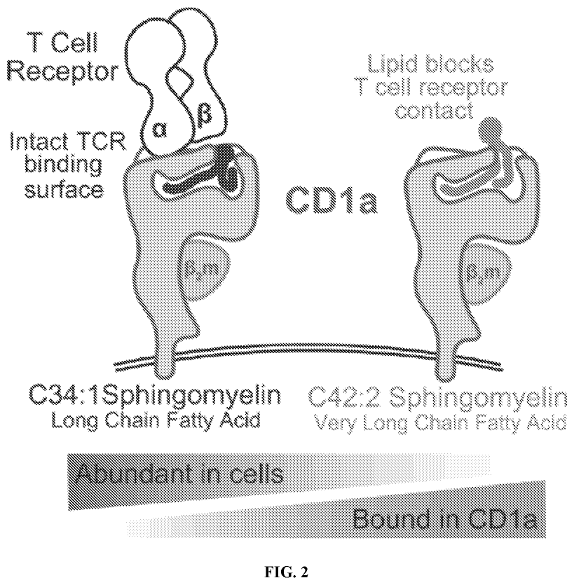

The compounds and methods of this disclosure advantageously block the binding and interaction between CD1 and lipid-reactive TCRs, thereby blocking the activation response of TCRs that directly bind to outer surface of CD1 proteins in the absence of any lipid antigen (See FIG. 2). In particular, the chemical design and synthetic production of antigens with longer and unsaturated alkyl chains allows stronger binding of synthetic analogs to CD1 proteins. Also, the longer alkyl chains position the hydrophilic head groups so that they protrude more extensively from the surface of CD1, which allows dominant negative inhibition of CD1 to many types TCRs that might otherwise bind to CD1.

The four types of CD1 proteins, CD1a, CD1b, CD1c and CD1d, are expressed on the major antigen presenting cell types found throughout the human body, including dendritic cells, B cells, macrophages and thymocytes. All four types of human CD1 proteins activate both αβ and γδ T cells from the blood, demonstrating systemic immune responses. Further, through study of human T cells, CD1d knockout mice or human CD1a transgenic mice, there is clear and credible evidence for CD1-mediated T cell response to viral, fungal and bacterial infections, as well as models of autoimmune diseases, anti-cancer response and T cell mediated drug interactions. Therefore, by measuring the molecular size, chemical structure, lipid length, and lipid saturation present in ligands isolated from each of the four human CD1 antigen presenting molecule types, data in this disclosure allows for discovery and synthetic optimization of ligands that bind to CD1a, CD1b, CD1c and CD1d, and by modulating the size of the lipid anchor or head group, synthetic compounds effectively blocking interaction of the CD1 isoform and a T cell can be prepared.

However, each type of CD1 protein has a distinctly shaped ligand binding groove. Each CD1 protein type shows distinct patterns of expression in the human body. For example, skin shows highest expression of CD1a, whereas gut and liver show highest expression of CD1d. All four human CD1 isoforms activate human T cells that circulate in the blood. The current examples discussed in this proposal emphasize one case in which CD1a is expressed at high levels in skin, and CD1a ligands are applied topically in skin. However, CD1b, CD1c and CD1d can also be expressed in human skin, and CD1a is expressed in other organs in the body. Thus, lipid ligands that bind any of the four types of human CD1 antigen presenting molecules (CD1a, CD1b, CD1c, CD1d) can be developed as immune regulators and applied using enteral, parenteral, topical, intradermal, mucosal or other routes.

T cell autoreactivity toward human CD1 proteins is known for human CD1a, CD1b, CD1c, and CD1d. The frequency of CD1a autoreactive T cells in the blood and skin is particularly high, when measured using limiting dilution cloning, single cell ELISA or tetramer assays. Lipid-loaded CD1 tetramers are oligomerized antigen presenting molecules that directly detect binding of TCRs to measure the rate of T cell response to any defined antigen or antigen presenting molecule. In skin, CD1a tetramer-binding T cells are about 100-fold more frequent than CD1b tetramer-binding T cells, and CD1a autoreactive tetramer+ T cells account for approximately 1% of total T cells on average and range to more than 20 percent in selected donors. Likewise, CD1a proteins are expressed at high density in skin, where they are found on myeloid dendritic cells and at particularly high density on Langerhans cells (LCs). Also, self-antigens for CD1a are extracted from skin in larger amounts than from other tissues.

The abundance of CD1a autoreactive T cells, CD1a protein and CD1a-presented antigens raises basic questions about possible mechanisms of inhibition of CD1a autoreactivity, which might otherwise result in autoimmune disease (See FIG. 2). Indeed, recent studies show that CD1a autoreactive T cells are present at higher levels in human skin involved with atopic dermatitis and psoriasis, as compared to skin from healthy donors or non-involved skin. Also, common allergens such as house dust mites contain phospholipase activity and generate lysolipids that bind to CD1a and contribute to T-intradermal cell response. In addition, squalene, which is a common lipid whose overproduction in acne syndromes (common acne, acne vulgaris), has been identified as a CD1a presented autoantigen. Further, transgenic expression of human CD1a in mouse skin results in marked augmentation of immune response to the contact dermatitis antigen, urushiol (poison ivy) as well as the TLR agonist imiquimod, which is widely used as a model of psoriasiform and dermatitic skin inflammation.

Against this background in which CD1a and skin lipids cause T cell mediated inflammation in the skin, the present disclosure provides compounds that specifically block interaction of a CD1a protein and T cell receptors, which in turn blocks CD1a-mediated T cell response or inflammation both in vitro and in vivo. Pharmaceutical compositions including these compounds (e.g., for topical application to skin) and methods of using these compounds and compositions, e.g., for treating skin inflammatory diseases are also provided. The compositions of matter and synthetic approaches for molecules described in this disclosure generate compounds that are both more homogenous and chemically different from the structures of endogenous lipids normally present in skin. The chemical structures present in novel synthetic sphingomyelins or sulfatides, which distinguish them from their natural counterparts in skin, account for optimal T cell inhibition. These proven approaches can be applied to other CD1 isoforms that bind distinct lipids and are expressed outside the skin.

### Compounds of Formula (I)

In some embodiments, the present application provides a compound of Formula (I):

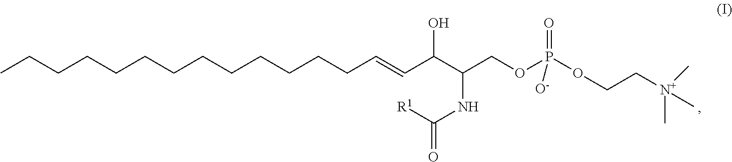

or a pharmaceutically acceptable salt thereof, wherein R¹ is as described herein.

In some embodiments, R¹ is C₁₉₋₃₇ alkenyl. In some embodiments, R¹ is C₂₀₋₄₁ alkenyl. In some embodiments, R¹ is C₂₁₋₃₇ alkenyl. In some embodiments, R¹ is C₂₂₋₄₁ alkenyl. In some embodiments, R¹ is C₂₃₋₃₇ alkenyl. In some embodiments, R¹ is C₂₄₋₄₁ alkenyl. In some embodiments, R¹ is C₂₅₋₃₇ alkenyl. In some embodiments, R¹ is C₂₆₋₄₁ alkenyl. In some embodiments, R¹ is C₂₇₋₃₇ alkenyl. In some embodiments, R¹ is C₂₈₋₄₁ alkenyl. In some embodiments, R¹ is C₂₉₋₃₇ alkenyl. In some embodiments, R¹ is C₃₀₋₄₁ alkenyl. In some embodiments, R¹ is C₃₁₋₃₇ alkenyl. In some embodiments, R¹ is C₃₂₋₄₁ alkenyl. In some embodiments, R¹ is C₂₇ alkenyl. In some embodiments, R¹ is C₂₈ alkenyl. In some embodiments, R¹ is C₂₉ alkenyl. In some embodiments, R¹ is C₃₀ alkenyl. In some embodiments, R¹ is C₃₁ alkenyl. In some embodiments, R¹ is C₃₂ alkenyl. In some embodiments, R¹ is C₃₃ alkenyl. In some embodiments, R¹ is C₃₄ alkenyl. In some embodiments, R¹ is C₃₅ alkenyl. In some embodiments, R¹ is C₃₆ alkenyl. In some embodiments, R¹ is C₃₇ alkenyl. In some embodiments, R¹ is C₃₈ alkenyl. In some embodiments, R¹ is C₄₀ alkenyl.

In some embodiments, any of said alkenyl groups comprises 2 double bonds. In some embodiments, the 2 double bonds are conjugated. In some embodiments, the 2 double bonds are not conjugated. In some embodiments, any of said alkenyl groups comprises 1 double bond.

In some embodiments, any of the double bonds is at C9, C11, C13, C15, C17, or C19 in the alkenyl group of R¹. In some embodiments, the double bond is at C9. In some embodiments, the double bond is at C11. In some embodiments, the double bond is at C13. In some embodiments, the double bond is at C15. In some embodiments, the double bond is at C17. In some embodiments, the double bond is at C19.

In some embodiments, any of the double bonds is Z configuration. In some embodiments, any of the double bonds is E configuration. In some embodiments, the alkenyl group of R¹ comprises 1 double bond in Z configuration. In some embodiments, the alkenyl group of R¹ comprises 1 double bond in E configuration. In some embodiments, the alkenyl group of R¹ comprises 1 double bond in Z configuration. In some embodiments, the alkenyl group of R¹ comprises 2 double bonds each in Z configuration. In some embodiments, the alkenyl group of R¹ comprises 2 double bonds each in E configuration. In some embodiments, the alkenyl group of R¹ comprises 1 double bond in E configuration and 1 double bond in Z configuration.

In some embodiments, the compound of Formula (I) has formula:

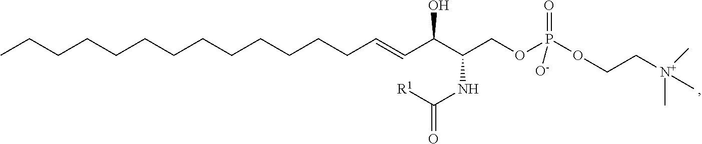

or a pharmaceutically acceptable salt thereof.

In some embodiments, the compound of Formula (I) has formula:

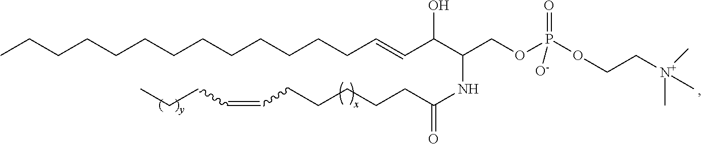

or a pharmaceutically acceptable salt thereof, wherein x and y are as described herein. In some embodiments, x is an integer from 3 to 13 and y is an integer from 6 to 26. In some embodiments, x is an integer from 3 to 13 and y is an integer from 16 to 26. In some embodiments, x is an integer from 3 to 13 and y is an integer from 6 to 16. In some embodiments, x is an integer from 3 to 13 and y is an integer from 8 to 16. In some embodiments, x is 3, 5, 7, 9, or 11. In some embodiments, x is 3. In some embodiments, x is 5. In some embodiments, x is 7. In some embodiments, x is 9. In some embodiments, x is 11. In some embodiments, y is from 0 to 26. In some embodiments, y is 0, 2, 4, 6, 8, 10, 12, 14, 16, 18, 20, 22, 24, or 26. In some embodiments, x is 3, and y is from 8 to 26. In some embodiments, x is 3 and y is from 16 to 26. In some embodiments, x is 5, and y is from 6 to 24. In some embodiments, x is 5 and y is from 14 to 24. In some embodiments, x is 7, and y is from 4 to 22. In some embodiments, x is 5 and y is from 12 to 22. In some embodiments, x is 9, and y is from 2 to 20. In some embodiments, x is 5 and y is from 10 to 20. In some embodiments, x is 11, and y is from 2 to 20. In some embodiments, the hydrocarbon chain comprising x and y comprises at least 19 and no more than 41 carbon atoms. In some embodiments, the hydrocarbon chain comprising x and y comprises at least 27 and no more than 41 carbon atoms.

In some embodiments, the compounds or Formula (I) are selected from any one of the following compounds in Table 1.

TABLE 1

| Cmpd | |
|---|---|
| No. | Chemical structure |
| 1 | 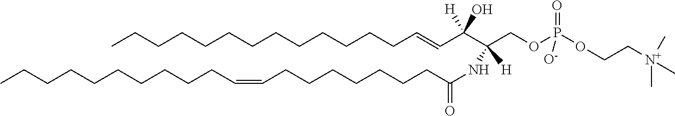 [SMILES: CCCCCCCCCC/C=C\CCCCCCCC(=O)N[C@@H](COP(=O)([O-])OCC[N+](C)(C)C)[C@H](O)/C=C/CCCCCCCCCCCCC] |
| 2 | 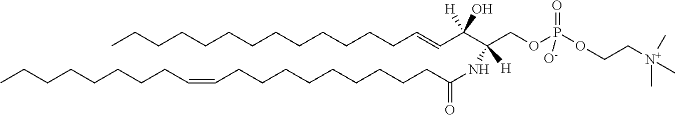 [SMILES: CCCCCCCC/C=C\CCCCCCCCCC(=O)N[C@@H](COP(=O)([O-])OCC[N+](C)(C)C)[C@H](O)/C=C/CCCCCCCCCCCCC] |
| 3 | 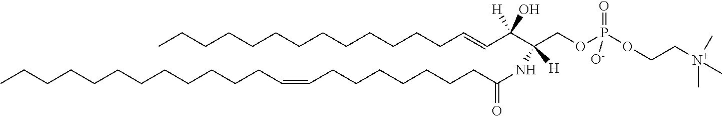 [SMILES: CCCCCCCCCCCC/C=C\CCCCCCCC(=O)N[C@@H](COP(=O)([O-])OCC[N+](C)(C)C)[C@H](O)/C=C/CCCCCCCCCCCCC] |
| 4 | 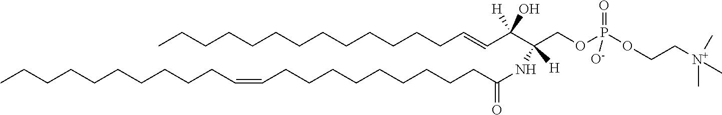 [SMILES: CCCCCCCCCC/C=C\CCCCCCCCCC(=O)N[C@@H](COP(=O)([O-])OCC[N+](C)(C)C)[C@H](O)/C=C/CCCCCCCCCCCCC] |
| 5 | 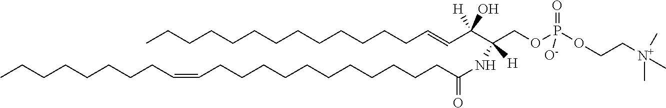 [SMILES: CCCCCCCC/C=C\CCCCCCCCCCCC(=O)N[C@@H](COP(=O)([O-])OCC[N+](C)(C)C)[C@H](O)/C=C/CCCCCCCCCCCCC] |
| 6 | 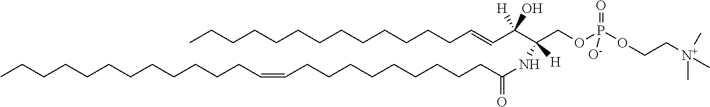 [SMILES: CCCCCCCCCCCC/C=C\CCCCCCCCCC(=O)N[C@@H](COP(=O)([O-])OCC[N+](C)(C)C)[C@H](O)/C=C/CCCCCCCCCCCCC] |
| 7 | 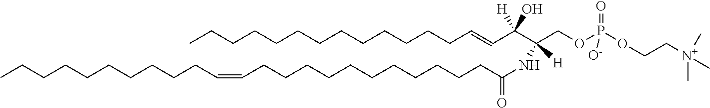 [SMILES: CCCCCCCCCC/C=C\CCCCCCCCCCCC(=O)N[C@@H](COP(=O)([O-])OCC[N+](C)(C)C)[C@H](O)/C=C/CCCCCCCCCCCCC] |
| 8 | 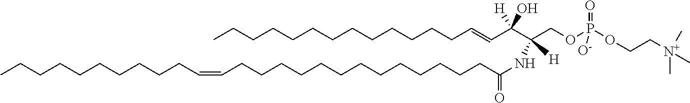 [SMILES: CCCCCCCCCC/C=C\CCCCCCCCCCCCCC(=O)N[C@@H](COP(=O)([O-])OCC[N+](C)(C)C)[C@H](O)/C=C/CCCCCCCCCCCCC] |
| 9 | 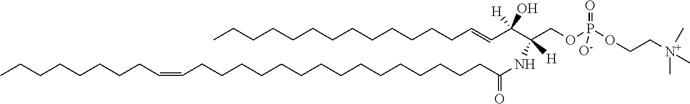 [SMILES: CCCCCCCC/C=C\CCCCCCCCCCCCCCCC(=O)N[C@@H](COP(=O)([O-])OCC[N+](C)(C)C)[C@H](O)/C=C/CCCCCCCCCCCCC] |
| 10 | 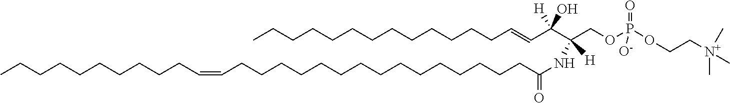 [SMILES: CCCCCCCCCC/C=C\CCCCCCCCCCCCCCCC(=O)N[C@@H](COP(=O)([O-])OCC[N+](C)(C)C)[C@H](O)/C=C/CCCCCCCCCCCCC] |

or a pharmaceutically acceptable salt of any of the foregoing.

In some embodiments the compound of Formula (I) has formula:

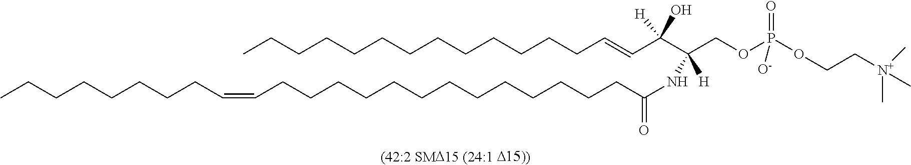 [SMILES: CCCCCCCC/C=C\CCCCCCCCCCCCCC(=O)N[C@@H](COP(=O)([O-])OCC[N+](C)(C)C)[C@H](O)/C=C/CCCCCCCCCCCCC]

or a pharmaceutically acceptable salt thereof.

In some embodiments, the compound of Formula (I) has formula:

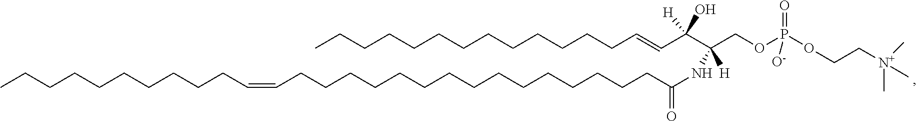 [SMILES: CCCCCCCCCC/C=C\CCCCCCCCCCCCCCCC(=O)N[C@@H](COP(=O)([O-])OCC[N+](C)(C)C)[C@H](O)/C=C/CCCCCCCCCCCCC]

or a pharmaceutically acceptable salt thereof.

In some embodiments, the present disclosure provides a compound selected from:

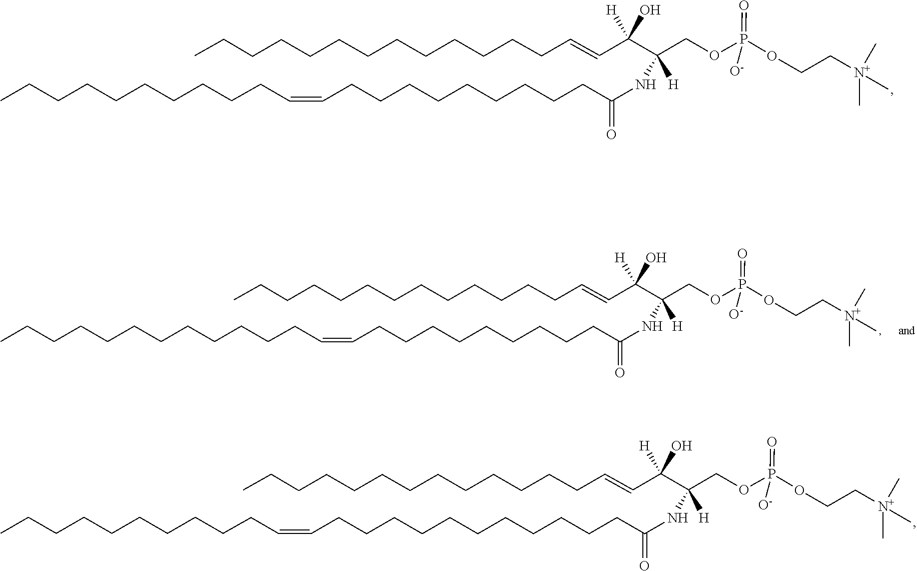

or a pharmaceutically acceptable salt of any of the foregoing.

### Compounds of Formula (II)

In some embodiments, the present application provides a compound of Formula (II):

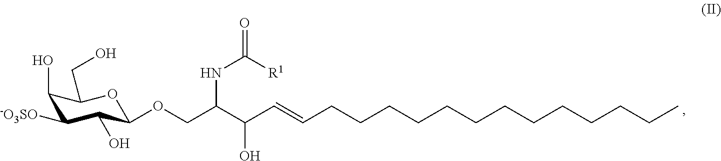

or a pharmaceutically acceptable salt thereof, wherein R¹ is as described herein.

In some embodiments, R¹ is C₁₉₋₃₇ alkenyl. In some embodiments, R¹ is C₂₀₋₄₁ alkenyl. In some embodiments, R¹ is C₂₁₋₃₇ alkenyl. In some embodiments, R¹ is C₂₂₋₄₁ alkenyl. In some embodiments, R¹ is C₂₃₋₃₇ alkenyl. In some embodiments, R¹ is C₂₄₋₄₁ alkenyl. In some embodiments, R¹ is C₂₅₋₃₇ alkenyl. In some embodiments, R¹ is C₂₆₋₄₁ alkenyl. In some embodiments, R¹ is C₂₇₋₃₇ alkenyl. In some embodiments, R¹ is C₂₈₋₄₁ alkenyl. In some embodiments, R¹ is C₂₉₋₃₇ alkenyl. In some embodiments, R¹ is C₃₀₋₄₁ alkenyl. In some embodiments, R¹ is C₃₁₋₃₇ alkenyl. In some embodiments, R¹ is C₃₂₋₄₁ alkenyl. In some embodiments, R¹ is C₂₇ alkenyl. In some embodiments, R¹ is C₂₈ alkenyl. In some embodiments, R¹ is C₂₉ alkenyl. In some embodiments, R¹ is C₃₀ alkenyl. In some embodiments, R¹ is C₃₁ alkenyl. In some embodiments, R¹ is C₃₂ alkenyl. In some embodiments, R¹ is C₃₃ alkenyl. In some embodiments, R¹ is C₃₄ alkenyl. In some embodiments, R¹ is C₃₅ alkenyl. In some embodiments, R¹ is C₃₆ alkenyl. In some embodiments, R¹ is C₃₇ alkenyl. In some embodiments, R¹ is C₃₈ alkenyl. In some embodiments, R¹ is C₄₀ alkenyl. In some embodiments, R¹ is C₁₉ alkenyl. In some embodiments, R¹ is C₂₀ alkenyl. In some embodiments, R¹ is C₂₁ alkenyl.

In some embodiments, R¹ is C₂₂ alkenyl. In some embodiments, R¹ is C₂₃ alkenyl. In some embodiments, R¹ is C₂₄ alkenyl. In some embodiments, R¹ is C₂₅ alkenyl. In some embodiments, R¹ is C₂₆ alkenyl. In some embodiments, R¹ is C₂₇ alkenyl. In some embodiments, R¹ is C₂₈ alkenyl. In some embodiments, R¹ is C₂₉ alkenyl. In some embodiments, R¹ is C₃₀ alkenyl. In some embodiments, R¹ is C₃₁ alkenyl. In some embodiments, R¹ is C₃₂ alkenyl. In some embodiments, R¹ is C₃₃ alkenyl. In some embodiments, R¹ is C₃₄ alkenyl. In some embodiments, R¹ is C₃₅ alkenyl. In some embodiments, R¹ is C₃₆ alkenyl. In some embodiments, R¹ is C₃₈ alkenyl. In some embodiments, R¹ is C₄₀ alkenyl.

In some embodiments, any of said alkenyl groups comprises 2 double bonds. In some embodiments, the 2 double bonds are conjugated. In some embodiments, the 2 double bonds are not conjugated. In some embodiments, any of said alkenyl groups comprises 1 double bond.

In some embodiments, any of the double bonds is at C9, C11, C13, C15, C17, or C19 in the alkenyl group of R¹. In some embodiments, the double bond is at C9. In some embodiments, the double bond is at C11. In some embodiments, the double bond is at C13. In some embodiments, the double bond is at C15. In some embodiments, the double bond is at C17. In some embodiments, the double bond is at C19.

In some embodiments, the alkenyl group of R¹ has one double bond at C9, C11, C13, C15, C17, or C19. In some embodiments, the alkenyl group of R¹ has double bonds at two of C9, C11, C13, C15, C17, and C19.

In some embodiments, any of the double bonds is Z configuration. In some embodiments, any of the double bonds is E configuration. In some embodiments, the alkenyl group of R¹ comprises 1 double bond in Z configuration. In some embodiments, the alkenyl group of R¹ comprises 1 double bond in E configuration. In some embodiments, the alkenyl group of R¹ comprises 1 double bond in Z configuration. In some embodiments, the alkenyl group of R¹ comprises 2 double bonds each in Z configuration. In some embodiments, the alkenyl group of R¹ comprises 2 double bonds each in E configuration. In some embodiments, the alkenyl group of R¹ comprises 1 double bond in E configuration and 1 double bond in Z configuration.

In some embodiments, the compound of Formula (II) has formula:

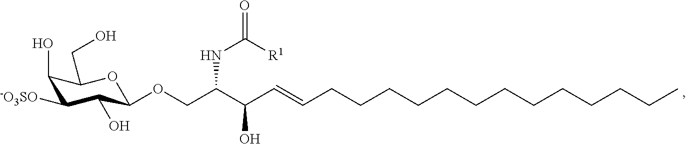

or a pharmaceutically acceptable salt thereof.

In some embodiments, the compound of Formula (II) has formula:

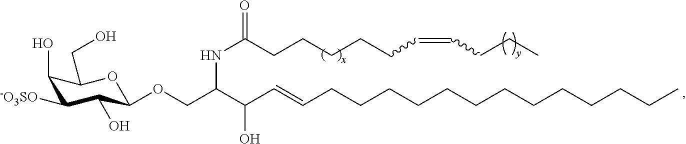

or a pharmaceutically acceptable salt thereof, wherein x and y are as described herein. In some embodiments, x is an integer from 3 to 13 and y is an integer from 6 to 26. In some embodiments, x is an integer from 3 to 13 and y is an integer from 16 to 26. In some embodiments, x is an integer from 3 to 13 and y is an integer from 6 to 16. In some embodiments, x is an integer from 3 to 13 and y is an integer from 8 to 16. In some embodiments, x is 3, 5, 7, 9, or 11. In some embodiments, x is 3. In some embodiments, x is 5. In some embodiments, x is 7. In some embodiments, x is 9. In some embodiments, x is 11. In some embodiments, y is from 0 to 26. In some embodiments, y is 0, 2, 4, 6, 8, 10, 12, 14, 16, 18, 20, 22, 24, or 26. In some embodiments, x is 3, and y is from 8 to 26. In some embodiments, x is 3 and y is from 16 to 26. In some embodiments, x is 5, and y is from 6 to 24. In some embodiments, x is 5 and y is from 14 to 24. In some embodiments, x is 7, and y is from 4 to 22. In some embodiments, x is 5 and y is from 12 to 22. In some embodiments, x is 9, and y is from 2 to 20. In some embodiments, x is 5 and y is from 10 to 20. In some embodiments, x is 11, and y is from 2 to 20. In some embodiments, the hydrocarbon chain comprising x and y comprises at least 19 and no more than 41 carbon atoms. In some embodiments, the hydrocarbon chain comprising x and y comprises at least 27 and no more than 41 carbon atoms.

In some embodiments, the compound of Formula (II) is selected from any one of the following compounds:

| Cmpd | |
|---|---|
| No. | Chemical structure |
| S1 | 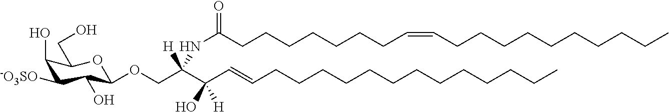 |
| S2 | 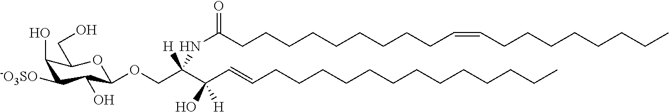 |
| S3 | 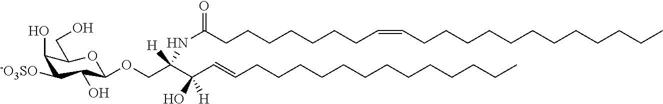 |
| S4 | 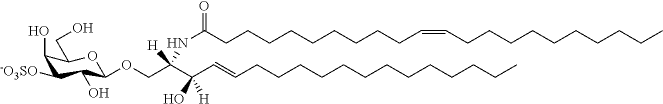 |
| S5 | 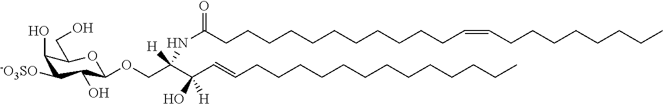 |
| S6 | 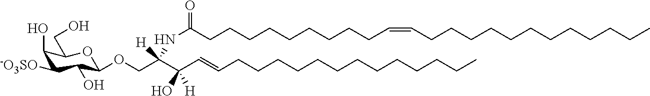 |
| S7 | 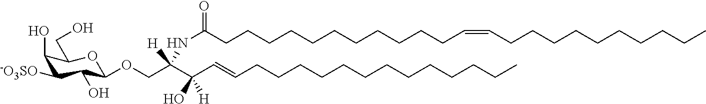 |
| S8 | 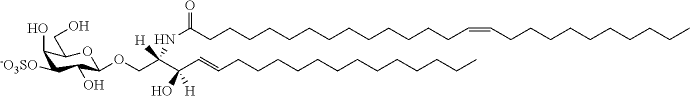 |
| S9 | 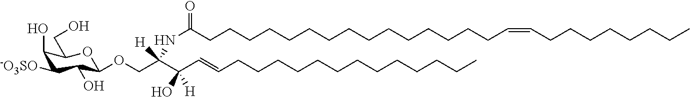 |
| S10 | 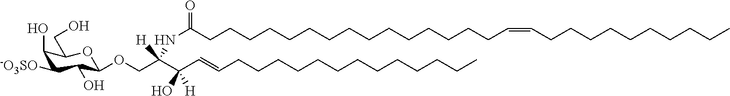 |

or a pharmaceutically acceptable salt of any of the foregoing.

In some embodiments, the compound of Formula (II) has formula:

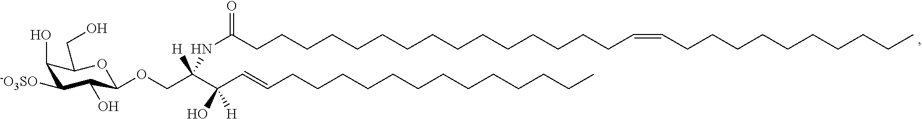

or a pharmaceutically acceptable salt thereof.

In some embodiments, the present disclosure provides a compound selected from:

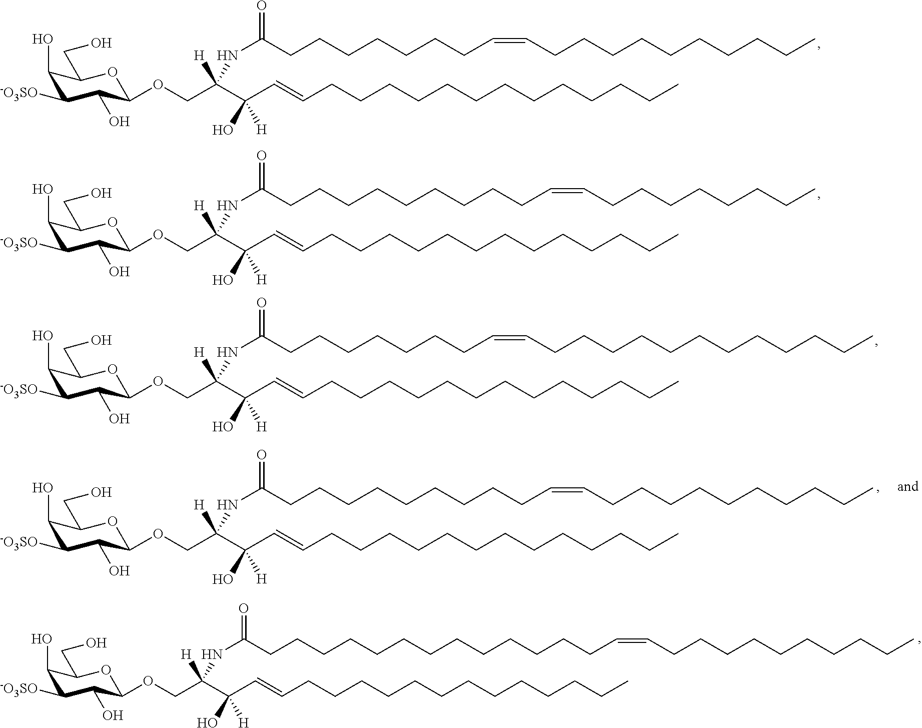

or a pharmaceutically acceptable salt of any of the foregoing.

### Pharmaceutically Acceptable Salts

In some embodiments, a salt of a compound of this disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (I) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (I) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of Formula (I) or Formula (II), or pharmaceutically acceptable salts thereof, are substantially isolated.

### Methods of Use

In one general aspect, the present disclosure provides a method of modulating interaction of a CD1 protein and a T cell, the method comprising contacting the CD1 protein with a compound as described herein, or a pharmaceutically acceptable salt thereof. In some embodiments, the modulating comprises inhibiting or blocking.

In some embodiments, the present disclosure provides a method of inhibiting interaction of a T cell and a CD1 protein, the method comprising contacting the CD1 protein on the surface of the cell with an effective amount of a compound as described herein, or a pharmaceutically acceptable salt thereof. In some embodiments, the present disclosure provides a method of inhibiting interaction of a T cell and a CD1 protein on a surface of another cell, the method comprising contacting the CD1 protein on the surface of the cell with an effective amount of a compound as described herein, or a pharmaceutically acceptable salt thereof. In some embodiments, the cell is in the tissue selected from epithelial tissue, mucosal tissue, connective tissue, muscle tissue, skin tissue, fibrous tissue, vascular tissue, and nervous tissue. In some embodiments, the cell is in the organ selected from lung, stomach, intestines, liver, thyroid, bladder, heart, eye, skin, kidney, gland, brain, pancreas, colon, lymph node, spleen, or prostate. In some embodiments, the method include a cell in the skin. Suitable examples of skin cells include any type of cell commonly found in skin, such as dendritic cells, B cells, macrophages, Langerhans cells, keratinocytes, melanocytes, Merkel cells, cells of the nerve endings, blood vessel cells, fibroblasts, adipocytes, mast cells, Schwann cells, or stem cells. In some embodiments, the skin cell is a dendric cell. In some embodiments, the skin cell is a Langerhans cell.

In some embodiments, the present disclosure provides a method of treating a disease, disorder, or condition responsive to inhibition of CD1 comprising administering to a subject in need thereof a therapeutically effective amount of a compound as described herein or a pharmaceutically acceptable salt thereof. In some embodiments, the CD1 protein is CD1a, CD1d, or CD1a and CD1d. In some embodiments, the CD1 protein is CD1a. In some embodiments, the CD1 protein is CD1d. In some embodiments, the CD1 protein is CD1a and CD1d.

In some embodiments, the disease, disorder or condition is an inflammatory disease chosen from pruritus, nodular prurigo, itch, atopic dermatitis, eczema, psoriasis, common acne, acne vulgaris, urticaria, seborrheic dermatitis, nummular dermatitis, dermatitis herpetiformis, poison ivy (urushiol) dermatitis, Balsam of Peru dermatitis, contact dermatitis of known or unknown origin, lupus erythematosus, drug reactions (cutaneous and systemic, including to sulfa antibiotics), Stevens-Johnson syndrome, toxic epidermal necrolysis, graft vs host disease, vasculitis, palmoplantar pustulosis, scleroderma, systemic sclerosis, autoimmune blistering conditions, vitiligo, alopecia areata, alopecia universalis, alopecia totalis, lichen planus, erythema multiforme, urticaria, other pruritic conditions, and other allergic and irritant skin diseases. In some embodiments, the contacting is carried out in vitro, in vivo, or ex vivo. In some embodiments, the present disclosure provides a method of inhibiting interaction of a T cell and a CD1 protein on a surface of a cell of a subject, the method comprising administering to the subject (e.g., in need thereof) a therapeutically effective amount of a compound as described herein, or a pharmaceutically acceptable salt thereof. In some embodiments, the cell is in any of the organs or tissues of the subject as described herein. In some embodiments, the cell is in the skin of the subject.

In some embodiments, the human CD1 protein is selected from CD1a, CD1b. CD1c, and CD1d. In some embodiments, the CD1 protein is CD1a. In some embodiments, the CD1 protein is CD1b. In some embodiments, the CD1 protein is CD1c. In some embodiments, the CD1 protein is CD1d. Without being bound by any particular theory or speculation, blocking the interaction of CD1 protein (e.g., CD1a protein) on a surface of a cell and a T cell results in treating inflammatory condition of the organ or tissue where the cell is located. In some embodiments, blocking the interaction of CD1 protein (e.g., CD1a protein) on a surface of a cell in a skin and a T cell results in treating inflammatory condition of the skin (e.g., dermatitis or psoriasis).

Accordingly, in some embodiments, the present disclosure provides a method of treating an inflammatory disease, the method comprising administering to a subject (e.g., in need thereof) a therapeutically effective amount of a compound as described herein, or a pharmaceutically acceptable salt thereof, or a pharmaceutical composition comprising same. In some embodiments, a CD1 protein (e.g., CD1a, CD1b, CD1c, CD1d, or a combination of the foregoing) is implicated in the pathology of the inflammatory disease. In some embodiments, the method comprises a step of identifying a subject in need of treatment for inflammatory disease or condition (e.g., inflammatory skin disease). For example, the step of identifying may include (i) diagnosing the subject with the inflammatory disease or condition or (ii) identifying the subject diagnosed with the inflammatory disease or condition. The subject may be diagnosed with inflammatory disease or condition by a physician (e.g., treating physician) or diagnostician. The subject may be diagnosed on the basis of a physical examination, laboratory testing, imaging, or any combination of the foregoing. For example, the subject may be diagnosed with an inflammatory disease on the basis of ex vivo blockade of T cells obtained from the organ or tissue of the subject (e.g., skin of the subject) by the lipid within the present claims. In another example, the subject may be diagnosed by testing for clinical response of the organ or tissue (e.g., skin) to the lipid treatment (e.g., “skin patch testing”).

In some embodiments, the inflammatory disease (e.g., in which CD1 is implicated in pathology) is selected from the group diabetes, rheumatoid arthritis, colitis, ulcerative colitis, asthma, allergic asthma, fatty liver disease, obesity, insulin resistance, inflammatory diseases of the liver and gut, steatohepatitis, liver inflammation, autoimmune diseases, atherosclerosis, hyperlipidemia, dyslipidemia, rhinitis, conjunctivitis, oral inflammation, thyroid disease, lung disease, inflammatory bowel disease (IBD), Crohn's disease, and chronic obstructive airways disease.

In some embodiments, the inflammatory disease (e.g., in which CD1 is implicated in pathology) is inflammatory skin disease. In some embodiments, the inflammatory skin disease is selected from atopic dermatitis, eczema, psoriasis, acne, acne vulgaris, urticaria, eczema, seborrheic dermatitis, nummular dermatitis, dermatitis herpetiformis, contact dermatitis of known or unknown origin, lupus erythematosus, drug reactions, Stevens-Johnson syndrome, toxic epidermal necrolysis, graft vs host disease, vasculitis, palmoplantar pustulosis, scleroderma, systemic sclerosis, autoimmune blistering conditions, vitiligo, alopecia areata, alopecia universalis, alopecia totalis, lichen planus, erythema multiforme, urticaria, and other pruritic conditions, and other allergic and irritant skin diseases. Examples of contact dermatitis include dermatitis from contact with skin creams, poison ivy, sulfa drugs, coenzyme Q, farnesol, balsam of Peru, or other contact allergens. Examples of inflammatory skin diseases also include skin inflammation caused by bacterial, fungal, or viral infections, or by an allergic reaction.

In some embodiments, the method of treating inflammatory skin condition includes administering the compound by applying a pharmaceutical formulation comprising the compound topically to the skin of the subject (e.g., as described herein).

In some embodiments, the disclosure provides a method of reducing T cell activation associated with an inflammatory disease, disorder, or condition comprising administering to a subject in need thereof a therapeutically effective amount of a compound as described herein or a pharmaceutically acceptable salt thereof.

In some embodiments, the disclosure provides a method of reducing expression of, activity of, and/or the release of a skin cytokine associated with an inflammatory disease, disorder, or condition comprising administering to a subject in need thereof a therapeutically effective amount of a compound as described herein or a pharmaceutically acceptable salt thereof. In some embodiments, the skin cytokine is chosen from IL1α, IFN-γ, IL-1β, IL-6, IL17α, IL-17f, TNFα, and IL-22. In some embodiments, the disclosure provides a method of reducing expression of, activity of, and/or the release of plasma cytokine associated with an inflammatory disease, disorder, or condition comprising administering to a subject in need thereof a therapeutically effective amount of a compound as described herein or a pharmaceutically acceptable salt thereof. In some embodiments, the plasma cytokine is chosen from IFN-β, IL-4, and IL17α. In some embodiments, the inflammatory disease, disorder, or condition is responsive to inhibition of CD1. In some embodiments, the inflammatory disease, disorder, or condition is associated with the skin. In some embodiments, the present disclosure provides a method of ameliorating one or more symptoms associated with any one of the inflammatory skin diseases described herein. Suitable examples of these symptoms include rashes, itching, burning, redness of skin, swelling, blistering, scaling, pimples, and cracking or thickening of the inflamed area.

### Compositions, Formulations, and Routes of Administration

The present application also provides pharmaceutical compositions comprising an effective amount of a compound of the present disclosure (e.g., Formula (I) or Formula (II)) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as ethanol, human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, subcutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, ocular, oral, parenteral, percutaneous, peridural, rectal, respiratory (inhalation), subcutaneous, sublingual, submucosal, topical, transdermal, transmucosal, transtracheal, ureteral, urethral and vaginal.

In some embodiments, the compounds described herein (e.g., compounds of Formula (I), Formula (II), or a pharmaceutically acceptable salt of any of the foregoing) can be administered topically, e.g., in a topical dosage form. In some embodiments, topical administration comprises administration to the skin. In some embodiments, topical administration is administration to the skin. The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application (e.g., skin). In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, cosmetics, leave-on exfoliants, prescription drugs, preservatives, scrub agents, silicones, skin-identical/repairing agents, slip agents, sunscreen actives, surfactants/detergent cleansing agents, penetration enhancers, alcohol (including but not limited to ethanol) and thickeners.

Compositions and formulations described herein may conveniently be presented in a unit dosage form, e.g., tablets, sustained release capsules, and in liposomes, and may be prepared by any methods well known in the art of pharmacy. Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid (such as ethanol); an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol, ethanol, and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

According to another embodiment, the present application provides an implantable drug release device impregnated with or containing a compound of this disclosure, or a composition comprising a compound of the present application and another therapeutic agent, such that said compound or therapeutic agent is released from said device and is therapeutically active. For example, the compound can be administered in a skin-integrated or a skin-adhesive wearable device, including cloth patches or “patch tests.”

### Dosages and Regimens

In the pharmaceutical compositions of the present application, a compound of the present disclosure (e.g., a compound of Formula (I) or Formula (II)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., Formula (I) or Formula (II)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) or Formula (II) is about 0.1 mg/kg, about 0.5 mg/kg, about 1 mg/kg, about 2 mg/kg, or about 5 mg/kg.

In some embodiments, when administered topically (e.g., in ethanol solution, cream, or ointment), an effective amount of the compound (e.g., Formula (I) or Formula (II)) can range, for example, from about 0.001 mg per cm² of the skin (mg/cm²) to about 50 mg/cm² (e.g., from about 0.01 mg/cm² to about 40 mg/cm²; from about 0.1 mg/cm² to about 30 mg/cm²; from about 0.5 mg/cm² to about 25 mg/cm²; from about 1 mg/cm² to about 20 mg/cm²; from about 1 mg/cm² to about 15 mg/cm²; from about 1 mg/cm² to about 10 mg/cm²; or from about 1 mg/cm² to about 5 mg/cm²). In some embodiments, an effective amount of a compound of Formula (I) or Formula (II) is about 0.5 mg/cm², about 1 mg/cm², about 2 mg/cm², about 5 mg/cm², or about 10 mg/cm².

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). For example, the compound can be administered by applying a thin layer of a topical formulation (such as ethanol, cream or ointment) to the skin of the patient once a day or as needed, until the symptoms (e.g., rash or itch) are reduced or completely ameliorated.

### Kits

The present disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Combinations

The compounds of the present disclosure can be used in combination with at least one medication or therapy useful, e.g., in treating or alleviating symptoms of inflammatory skin disease. Suitable examples of such medications include corticosteroids, calcineurin inhibitors, or antibacterial or antifungal agents if the skin inflammation was caused by infection. For example, the additional agent may be hydrocortisone, cortisone, prednisone, betamethasone, tixocortol, prednisolone, methylprednisolone, amcinonide, budesonide, desonide, fluocinolone acetonide, fluocinonide, halcinonide, triamcinolone acetonide, beclometasone, dexamethasone, fluocortolone, halometasone, mometasone, clobetasol, halobetasol, diflorasone, or fluticasone, or a pharmaceutically acceptable salt thereof. The second agent may also be an NSAID (e.g., administered orally), such as ibuprofen, naproxen, diclofenac, celecoxib, mefenamic acid, etoricoxib, aspirin, or indomethacin, or a pharmaceutically acceptable salt thereof. The compound of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

### Definitions

As used herein, the term “about” means “approximately” (e.g., plus or minus approximately 10% of the indicated value).

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, or C₆ alkyl group in the alternative. The term “C₁₋₆ alkyl” does not encompass a mixture of compounds with variable alkyl groups within the indicated range.

As used herein, “Cn-m alkenyl” refers to an alkyl group having one or more double carbon-carbon bonds and having n to m carbons. The “alkyl” refers to hydrocarbon group that may be straight-chain or branched, including isomethyl or anteisomethyl branched alkyls. For example, the alkyl group may include one double carbon-carbon bond. In another example, the alkyl group may include two double carbon-carbon bonds (e.g., conjugated or unconjugated). In some examples, n can be from 19 to 60, from 19 to 40, from 19 to 37, from 20 to 60, from 20 to 40, from 20 to 37, from 21 to 60, from 21 to 40, from 21 to 37, from 22 to 60, from 22 to 40, from 22 to 37, from 23 to 60, from 23 to 40, from 23 to 37, from 23 to 60, from 23 to 40, from 23 to 37, from 24 to 60, from 24 to 40, from 24 to 37, from 25 to 60, from 25 to 40, from 25 to 37, from 26 to 60, from 26 to 40, from 26 to 37, from 27 to 60, from 27 to 40, from 27 to 37, from 28 to 60, from 28 to 40, from 28 to 37, from 29 to 60, from 29 to 40, from 29 to 37, from 30 to 60, from 30 to 40, or from 30 to 37. In some examples, m can be from 28 to 60, from 28 to 40, from 28 to 37, from 29 to 60, from 29 to 40, from 29 to 37, from 30 to 60, from 30 to 40, from 30 to 37, from 31 to 60, from 31 to 40, from 31 to 37, from 32 to 60, from 32 to 40, from 32 to 37, from 33 to 60, from 33 to 40, from 33 to 37, from 34 to 60, from 34 to 40, from 34 to 37, from 35 to 60, from 35 to 40, or from 35 to 37. While the alkyl length for any given formulation can vary as outlined above based on the CD1 isoform and tissue targeted, individual therapeutic doses will typically contain compounds with only one kind of optimized alkyl chain length.

The term “compound” as used herein is meant to include all stereoisomers, geometric isomers, and tautomers of the structures depicted, as well is isotopic variants, such as those including one or more isotopes at higher than natural abundance (e.g., deuterium and/or ¹³C). Compounds herein identified by name or structure as one particular tautomeric form are intended to include other tautomeric forms unless otherwise specified. Known CD1a ligands can have isomethyl or anteisomethyl branched alkane structures that promote CD1a binding.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present disclosure. Cis and trans geometric isomers of the compounds of the present disclosure are described and may be isolated or synthesized as a mixture of isomers or as separated isomeric forms. In some embodiments, the compound has the (R)-configuration. In some embodiments, the compound has the(S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the CD1 protein with a compound of the disclosure includes the administration of a compound of the present disclosure to an individual or patient, such as a human, having CD1 protein, as well as, for example, introducing a compound of the disclosure into a sample containing a cellular or purified preparation containing the CD1.

As used herein, the term “individual,” “patient,” or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

### EXAMPLES

Many published papers have previously identified naturally occurring CD1 proteins and ligands, including natural ligands that block CD1 interaction with T cell receptors. The exemplified compounds herein are synthetically engineered non-natural lipids that block T cell receptor binding and T cell inflammatory response.

### Example 1—Synthesis of Compounds

General Procedures for Chemical Analyses: 1H NMR spectra were recorded at 400 MHz or 300 MHz, using Bruker AV 400 and Bruker AV 300 spectrometers. Proton-decoupled ¹³C NMR spectra were recorded at 100 MHz using a Bruker AV 400 spectrometer. Chemical shifts are reported as δ values (ppm) referenced to the following solvent signals: CHCl₃, δ_{H} 7.26; CDCl₃, δ_{C} 77.0; CH₃OH, δ_{H} 3.34; CD₃OD, δ_{C} 49.9. When a 2:1 mixture of CDCl₃ and CD₃OD was used as solvent, chemical shifts are reported referenced to the following solvent signals: CH₃OH, δ_{H} 3.34; CD₃OD, δ_{C} 49.9. Mass spectra were recorded on a liquid chromatography time-of-flight (LCT) spectrometer utilizing electrospray ionization with a methanol mobile phase and are reported as (m/z (%)). HRMS were recorded on a Micromass LCT spectrometer using a lock mass incorporated into the mobile phase. All reagents were obtained from commercial sources and were used without further purification, unless stated otherwise. Anhydrous solvents were purchased from Sigma-Aldrich, UK, and stored over 4 Å molecular sieves and under an argon atmosphere. Analytical thin layer chromatography (TLC) was performed on aluminium plates pre-coated with Merck silica gel 60A F-254 as adsorbent. The developed plates were air-dried, visualized by UV detection (at 254 nm) and/or stained with 5% phosphomolybdic acid in EtOH (MPA spray). Compounds were purified by flash column chromatography on Merck silica gel (particle size 40-63 μm mesh) or Fluka 60 (40-60 μm mesh) silica gel.

The compounds of Formula I of the disclosure can be prepared according to Scheme 1 and further described below.

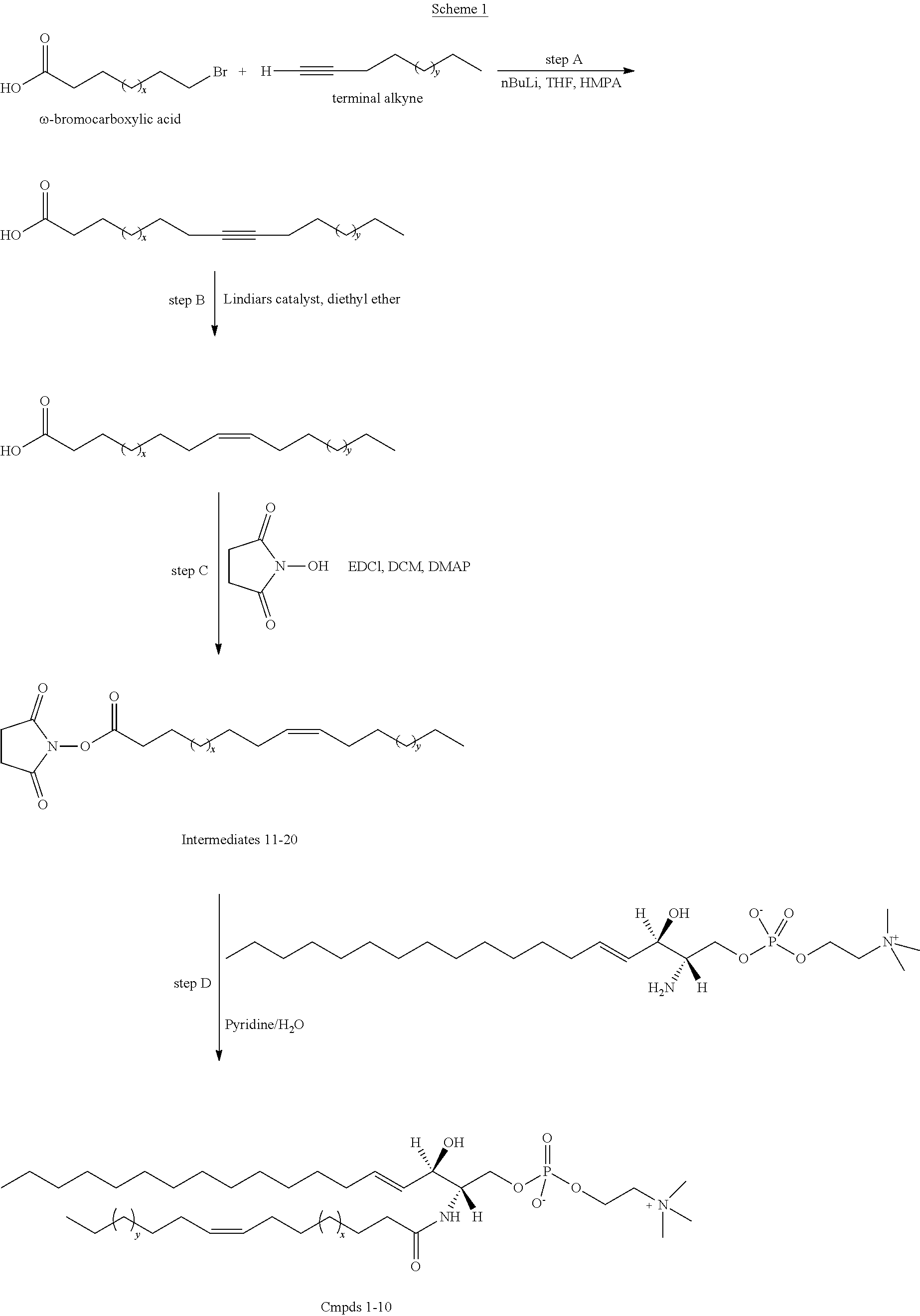

Compounds 1-10 have the following structural olefinic characteristics:

| | |
|---|---|
| Cmpd 1. | 38:2 SM d18:1/20:1 (9Z), x = 3, y = 6 |
| Cmpd 2. | 38:2 SM d18:1/20:1 (11Z) x = 5, y = 4 |
| Cmpd 3. | 40:2 SM d18:1/22:1 (9Z) x = 3, y = 8 |
| Cmpd 4. | 40:2 SM d18:1/22:1 (11Z) x = 5, y = 6 |
| Cmpd 5. | 40:2 SM d18:1/22:1 (13Z) x = 7, y = 4 |
| Cmpd 6. | 42:2 SM d18:1/24:1 (11Z) x = 5, y = 8 |
| Cmpd 7. | 42:2 SM d18:1/24:1 (13Z) x = 7, y = 6 |
| Cmpd 8. | 44:2 SM d18:1/26:1 (15Z) x = 9, y = 6 |
| Cmpd 9. | 44:2 SM d18:1/26:1 (17Z) x = 11, y = 4 |
| Cmpd 10. | 46:2 SM d18:1/28:1 (17Z) x = 11, y = 6 |

Step A: General Procedure for the C-Alkylation of Acetylide Anions with □n-Bromocarboxylic Acids to Yield Chain-Extended Carboxylic Acids with Internal Alkyne Group (-ynoic Acids)

n-Butyl lithium (2.5 M hexane solution, 2.5 mmol, 2.5 eq) was added dropwise to a stirring solution of the terminal alkyne (1 mmol) in anhydrous tetrahydrofuran at −78° C. under an Ar atmosphere. The reaction mixture was allowed to warm up to −20° C. over an hour. A solution of the respective ω-bromocarboxylic acids (0.8 mmol, 0.8 eq) in a mixture of HMPA:THF (1:5) (10 ml) was then added dropwise over 10 minutes at −20° C. The reaction mixture was allowed to gradually warm up to room temperature and stirred overnight. The reaction was then cooled to 0° C. before being quenched by adding saturated aqueous ammonium chloride (20 mL). The biphasic mixture was stirred vigorously for 10 minutes and then taken up in H₂O (100 mL). The resulting mixture was extracted with EtOAc (3×50 mL). The combined organic layers were washed with brine (50 mL), dried over anhydrous Na₂SO₄ and concentrated in vacuo. The residue was purified by flash chromatography using hexanes:EtOAc (3:1) as eluent to yield the -ynoic acid.

Step B: General Procedure for the Hydrogenation of the Internal Alkyne in the -ynoic Acid to Yield Internal Cis-Alkene in the -enoic Acid.

Lindlar's catalyst (0.25 mmol, 0.25 eq) was added to a solution of the -ynoic acids (1 mmol) in anhydrous diethyl ether (50 mL) containing 0.5 mL of quinoline. The reaction mixture was degassed, flushed with H₂ gas and allowed to stir under a H₂ atmosphere overnight. The mixture was then filtered over Celite and the filtrate was concentrated in vacuo to give a residue that was purified by flash chromatography using EtOAc (3:1) as eluent to yield the -enoic acid.

Step C: General Procedure for the Synthesis of N-Hydroxysuccinimide (NHS) Ester of the -enoic Acids (Intermediates 11-20).

Ethyl-3-(3-dimethylaminopropyl) carbodiimide (EDCI) (105 mg, 0.55 mmol, 1.1 eq), N-hydroxysuccinimide (70 mg, 0.60 mmol, 1.2 eq) and 4-dimethylaminopyridine (10% of acid weight) were added to a solution of the -enoic acid (0.50 mmol) in anhydrous DCM (10 mL). The resulting solution was heated at 40° C. for 4 h. Upon completion of the reaction, the solution was diluted with DCM (20 mL) and washed sequentially with H₂O (20 mL) and brine (20 mL). The organic layer was dried over anhydrous Na₂SO₄ and concentrated in vacuo. The residue was purified by flash chromatography using EtOAc (4:1) as eluent to yield the NHS activated-enoic acid.

Synthesis of intermediate 11

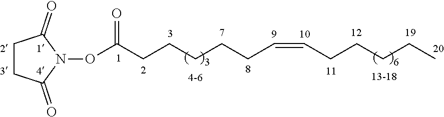

Prepared following general procedures 1, 2 and 3 using 1-dodecyne (168 mg, 1 mmol) and 8-bromooctanoic acid (180 mg, 0.8 mmol, 0.8 eq.). The title compound was obtained as an off-white foam after purification (196 mg, 60%). ¹H NMR (CDCl₃): δ 5.30-5.39 (H-9, H-10, 2H, t, J=5.6 Hz), 2.79-2.88 (H-2′, H-3′, 4H, m), 2.56-2.64 (H-2, 2H, m), 1.15-2.06 (H-3-H-8, H-11-H-19, 30H, m), 0.83-0.91 (H-20, 3H, t, J=6.9 Hz). 13C NMR (100 MHz, CDCl₃): 128.70, 128.62 (C-9, C-10), 28.55-32.81 (C-2′, C-3′, C-2, C-8, C-11), 23.33-29.70 (multiple signals C3-C-8, C-12-C-19), 12.87 (C-20); MS (TOF ES+) m/z 430.3 ([M+Na]+,100%); HRMS (TOF ES+) calcd for C₂₄H₄₁NO₄[M+Na]⁺: 430.3036, found 430.3039.

### Synthesis of Intermediate 12.

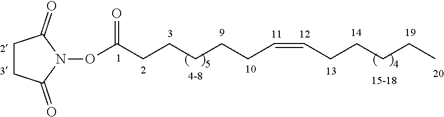

Prepared following general procedures 1, 2 and 3 using 1-decyne (139 g, 1 mmol) and 10-bromodecanoic acid (205 mg, 0.8 mmol, 0.8 eq.). The title compound was obtained as an off-white foam after purification (212 mg, 65%). ¹H NMR (CDCl₃): δ 5.28-5.39 (H-11, H-12, 2H, t, J=5.7 Hz), 2.75-2.91 (H-2′, H-3′, 4H, m), 2.55-2.65 (H-2, 2H, m), 1.15-2.06 (H-3-H-10, H-13-H-19, 30H, m), 0.83-0.90 (H-20, 3H, t, J=6.9 Hz). ¹³C NMR (100 MHz, CDCl₃): 130.60, 129.72 (C-11, C-12), 28.65-31.95 (C-2′, C-3′, C-2, C-10, C-13), 23.03-29.50 (multiple signals C3-C-9, C-14-C-19), 12.17 (C-20); MS (TOF ES+) m/z 430.3 ([M+Na]+,100%); HRMS (TOF ES+) calcd for C₂₄H₄₁NO₄[M+Na]⁺: 430.3036, found 430.3033.

### Synthesis of Intermediate 13

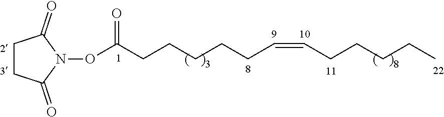

Prepared following general procedures 1, 2 and 3 using 1-tetradecyne (196 mg, 1 mmol, 4.0 eq.) and 8-bromooctanoic acid (175 mg, 0.8 mmol, 0.8 eq.). The title compound was obtained as an off-white foam after purification (210 mg, 60%). ¹H NMR (CDCl₃): δ 5.35 (H-9, H-10, 2H, t, J=5.8 Hz), 2.66-2.89 (H-2′, H-3′, 4H, m), 2.53-2.66 (H-2, 2H, m), 1.05-2.16 (H-3-H-7, H-12-H-21, 34H, m), 0.85 (H-22, 3H, t, J=7.1 Hz). 13C NMR (100 MHz, CDCl₃): 132.30, 131.72 (C-9, C-10), 28.55-32.16 (C-2′, C-3′, C-2, C-8, C-11), 23.05-26.80 (multiple signals C3-C-7, C-12-C-21), 12.33 (C-22); MS (TOF ES+) m/z 458.3 ([M+Na]+,100%); HRMS (TOF ES+) calcd for C₂₆H₄₅NO₄[M+Na]⁺: 458.3349, found 458.3345.

### Synthesis of Intermediate 14

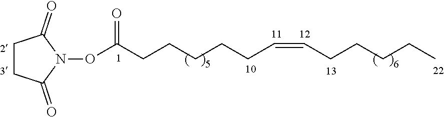

Prepared following general procedures 1, 2 and 3 using 1-dodecyne (163 mg, 1 mmol) and 10-bromodecanoic acid (196 mg, 0.8 mmol, 0.8 eq.). The title compound was obtained as an off-white foam after purification (198 mg, 56%). ¹H NMR (CDCl₃): δ 5.13 (H-11, H-12, 2H, t, J=6.2 Hz), 2.65-2.93 (H-2′, H-3′, 4H, m), 2.58-2.72 (H-2, 2H, m), 1.02-2.18 (H-3-H-9, H-14-H-21, 34H, m), 0.88 (H-22, 3H, t, J=6.9 Hz). 13C NMR (100 MHz, CDCl₃): 131.7, 130.12 (C-11, C-12), 28.56-33.05 (C-2′, C-3′, C-2, C-10, C-13), 23.01-26.53 (multiple signals C3-C-9, C-14-C-21), 11.93 (C-22); MS (TOF ES+) m/z 458.3 ([M+Na]+,100%); HRMS (TOF ES+) calcd for C₂₆H₄₅NO₄[M+Na]⁺: 458.3349, found 458.3352.

### Synthesis of Intermediate 15

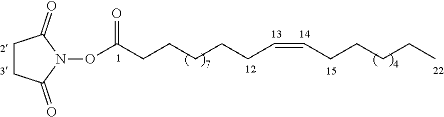

Prepared following general procedures 1, 2 and 3 using 1-decyne (141 mg, 1 mmol) and 12-bromododecanoic acid (221 mg, 0.8 mmol, 0.8 eq.). The title compound was obtained as an off-white foam after purification (223 mg, 65%). ¹H NMR (CDCl₃): δ 5.21 (H-13, H-14, 2H, t, J=5.9 Hz), 2.56-2.87 (H-2′, H-3′, 4H, m), 2.55-2.70 (H-2, 2H, m), 1.13-2.21 (H-3-H-11, H-16-H-21, 34H, m), 0.89 (H-22, 3H, t, J=6.7 Hz). 13C NMR (100 MHz, CDCl₃): 132.17, 131.88 (C-13, C-14), 28.67-32.78 (C-2′, C-3′, C-2, C-12, C-15), 23.11-27.02 (multiple signals C3-C-11, C-16-C-21), 12.13 (C-22); MS (TOF ES+) m/z 458.3 ([M+Na]+,100%); HRMS (TOF ES+) calcd for C₂₆H₄₅NO₄[M+Na]⁺: 458.3349, found 458.3351.

### Synthesis of Intermediate 16

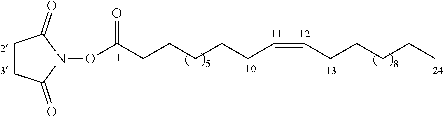

Prepared following general procedures 1, 2 and 3 using 1-tetradecyne (198 mg, 1 mmol) and 10-bromodecanoic acid (203 mg, 0.8 mmol, 0.8 eq.). The title compound was obtained as a white foam after purification (212 mg, 57%). ¹H NMR (CDCl₃): δ 5.36 (H-11, H-12, 2H, t, J=5.6 Hz), 2.68-2.72 (H-2′, H-3′, 4H, m), 2.53 (H-2, 2H, t, J-7.4 Hz), 1.98-2.07 (H-10, H-13, 4H, m), 1.08-1.72 (H-3-H-19, H-14-H-23, 34 H, m), 0.80 (H-24, 3H, t, J-6.9 Hz). 13C NMR (100 MHz, CDCl₃): 128.13, 128.02, (C-11, C-12), 28.01-35.61 (C-2′, C-3′, C-2, C-10, C-13), 18.05-27.12 (multiple signals C3-C-9, C-14-C-23), 13.12 (C-24); MS (TOF ES+) m/z 486.4 ([M+Na]+,100%); HRMS (TOF ES+) calcd for C₂₈H₄₉NO₄[M+Na]⁺: 486.3662, found 486.3665.

### Synthesis of Intermediate 17

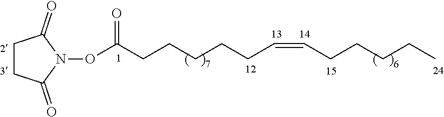

Prepared following general procedures 1, 2 and 3 using 1-dodecyne (165 mg, 1 mmol) and 12-bromododecanoic acid (220 mg, 0.8 mmol, 0.8 eq.). The title compound was obtained as a white foam after purification (231 mg, 62%). ¹H NMR (CDCl₃): δ 5.51 (H-13, H-14, 2H, t, J=5.7 Hz), 2.66-2.71 (H-2′, H-3′, 4H, m), 2.57 (H-2, 2H, t, J-7.4 Hz), 2.11-2.25 (H-12, H-15, 4H, m), 1.05-1.70 (H-3-H-11, H-16-H-23, 34 H, m), 0.82 (H-24, 3H, t, J=7.2 Hz). ¹³C NMR (100 MHz, CDCl₃): 128.11, 127.97, (C-13, C-14), 28.00-35.55 (C-2′, C-3′, C-2, C-12, C-15), 17.98-27.11 (multiple signals C3-C-11, C-16-C-23), 13.17 (C-24); MS (TOF ES+) m/z 486.4 ([M+Na]+,100%); HRMS (TOF ES+) calcd for C₂₈H₄₉NO₄[M+Na]⁺: 486.3662, found 486.3659.

### Synthesis of Intermediate 18

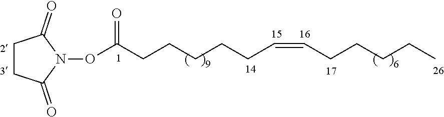

Prepared following general procedures 1, 2 and 3 using 1-dodecyne (168 mg, 1 mmol) and 14-bromotetradeanoic acid (250 mg, 0.8 mmol, 0.8 eq.). The title compound was obtained as a white powder after purification (252 mg, 64%). ¹H NMR (CDCl₃): δ 5.38 (H-15, H-16, 2H, t, J-5.6 Hz), 2.70-2.76 (H-2′, H-3′, 4H, m), 2.55 (H-2, 2H, t, J-7.5 Hz), 2.02-2.15 (H-14, H-17, 4H, m), 1.10-1.72 (H-3-H-13, H-18-H-25, 38 H, m), 0.80 (H-26, 3H, t, J=7.1 Hz). ¹³C NMR (100 MHz, CDCl₃): 128.61, 128.12, (C-17, C-18), 28.05-35.12 (C-2′, C-3′, C-2, C-14, C-17), 18.55-27.92 (multiple signals C3-C-13, C-18-C-25), 13.51 (C-26); MS (TOF ES+) m/z 514.39 ([M+Na]+,100%); HRMS (TOF ES+) calcd for C₃₀H₅₃NO₄[M+Na]⁺: 514.3975, found 514.3972.

### Synthesis of Intermediate 19

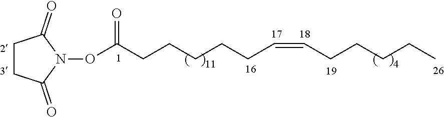

Prepared following general procedures 1, 2 and 3 using 1-decyne (136 mg, 1 mmol) and 16-bromohexadecanoic acid (270 mg, 0.8 mmol, 0.8 eq.). The title compound was obtained as a white powder after purification (245 mg, 59%). ¹H NMR (CDCl₃): δ 5.30-5.39 (H-17, H-18, 2H, t, J=5.6 Hz), 2.71-2.82 (H-2′, H-3′, 4H, m), 2.53 (H-2, 2H, t, J=7.5 Hz), 2.08-2.22 (H-16, H-19, 4H, m), 1.12-1.75 (H-3-H-15, H-20-H-25, 38 H, m), 0.81 (H-26, 3H, t, J=6.9 Hz). 13C NMR (100 MHz, CDCl₃): 128.70, 128.62, (C-17, C-18), 28.55-35.02 (C-2′, C-3′, C-2, C-16, C-19), 18.53-28.1 (multiple signals C3-C-15, C-20-C-25), 13.47 (C-26); MS (TOF ES+) m/z 514.39 ([M+Na]+,100%); HRMS (TOF ES+) calcd for C₃₀H₅₃NO₄[M+Na]⁺: 514.3975, found 514.3977.

### Synthesis of Intermediate 20

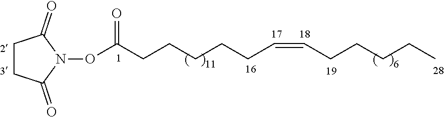

Prepared following general procedures 1, 2 and 3 using 1-dodecyne (164 mg, 1 mmol) and 16-bromohexadecanoic acid (266 mg, 0.8 mmol, 0.8 eq.). The title compound was obtained as an off-white powder after purification (245 mg, 59%). ¹H NMR (CDCl₃): δ 5.30-5.39 (H-17, H-18, 2H, t, J=5.4 Hz), 2.82-2.86 (H-2′, H-3′, 4H, m), 2.60-2.65 (H-2, 2H, t, J=7.5 Hz), 1.98-2.10 (H-16, H-19, 4H, m), 1.05-1.54 (H-3-H-15, H-20-H-27, 42H, m), 0.82-0.89 (H-28, 3H, t, J=6.9 Hz). ¹³C NMR (100 MHZ, CDCl₃): 127.80, 128.82 (C-17, C-18), 28.75-33.18 (C-2′, C-3′, C-2, C-16, C-19), 20.53-27.90 (multiple signals C3-C-15, C-20-C-27), 12.87 (C-28); MS (TOF ES+) m/z 537.46 ([M+NH₄]+,100%); HRMS (TOF ES+) calcd for C₃₂H₅₇NO₄[M+NH₄]⁺: 537.4631, found 537.4637.
Step D: General Procedure for the Acylation of Lyso-Sphingomyelin with the NHS Ester of the -enoic Acids from General Procedure (3) to Yield the Desired Sphingomyelin Compounds 1-10.

To a solution of lyso sphingomyelin (20 mg, 0.04 mmol) in pyridine:H₂O (9:1, 2 mL) the NHS activated-enoic acid (0.08 mmol, 2 eq) was added and the solution stirred overnight at 60° C. Upon completion of the reaction, indicated by TLC analysis, the reaction mixture was concentrated under vacuo to give a residue that was purified by flash chromatography (gradient from CHCl₃ to 35% MeOH in CHCl₃) to yield the acylated target compound as a white solid.

### Synthesis of Compound 1

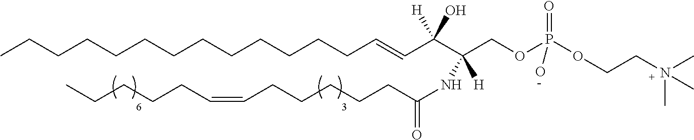

Prepared following general procedure 4 using lyso-sphingomyelin (20 mg, 0.04 mmol) and intermediate 11 (33 mg, 0.08 mmol, 2 eq). The title compound was obtained as an off-white powder after purification (22 mg, 73%). MS (TOF ES+) m/z 757.6 ([M+H]+,100%); HRMS (TOF ES+) calcd for C₄₃H₈₅N₂O₆P[M+H]⁺: 757.6145, found 757.6147.

### Synthesis of Compound 2

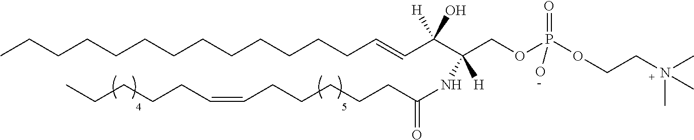

Prepared following general procedure 4 using lyso-sphingomyelin (20 mg, 0.04 mmol) and intermediate 12 (31 mg, 0.08 mmol, 2 eq). The title compound was obtained as an off-white powder after purification (19 mg, 62%). MS (TOF ES+) m/z 757.6 ([M+H]+, 100%); HRMS (TOF ES+) calcd for C₄₃H₈₅N₂O₆P[M+H]⁺: 757.6145, found 757.6142.

### Synthesis of Compound 3

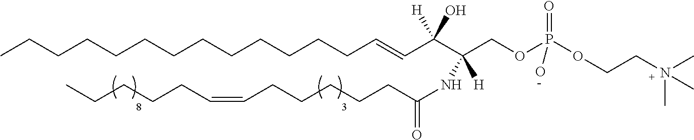

Prepared following general procedure 4 using lyso-sphingomyelin (20 mg, 0.04 mmol) and intermediate 13 (35 mg, 0.08 mmol, 2 eq). The title compound was obtained as an off-white powder after purification (21 mg, 67%). MS (TOF ES+) m/z 785.7 ([M+H]+, 100%); HRMS (TOF ES+) calcd for C₄₅H₈₉N₂O₆P[M+H]⁺: 785.6458, found 785.6455.

### Synthesis of Compound 4

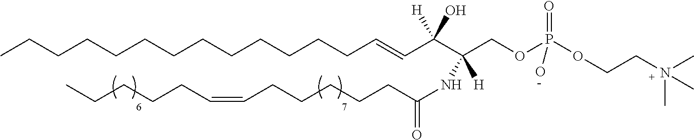

Prepared following general procedure 4 using lyso-sphingomyelin (20 mg, 0.04 mmol) and intermediate 14 (35 mg, 0.08 mmol, 2 eq). The title compound was obtained as an off-white powder after purification (20 mg, 63%). MS (TOF ES+) m/z 785.7 ([M+H]+, 100%); HRMS (TOF ES+) calcd for C₄₅H₈₉N₂O₆P[M+H]⁺: 785.6458, found 785.6460.

### Synthesis of Compound 5

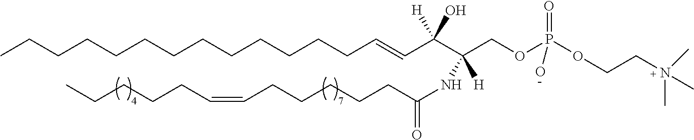

Prepared following general procedure 4 using lyso-sphingomyelin (20 mg, 0.04 mmol) and intermediate 15 (35 mg, 0.08 mmol, 2 eq). The title compound was obtained as an off-white powder after purification (18 mg, 57%). MS (TOF ES+) m/z 785.7 ([M+H]+, 100%); HRMS (TOF ES+) calcd for C₄₅H₈₉N₂O₆P[M+H]⁺: 785.6458, found 785.6462.

### Synthesis of Compound 6

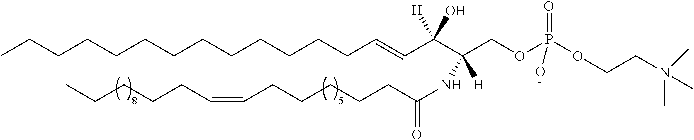

Prepared following general procedure 4 using lyso-sphingomyelin (20 mg, 0.04 mmol) and intermediate 16 (37 mg, 0.08 mmol, 2 eq). The title compound was obtained as an off-white powder after purification (22 mg, 67%). MS (TOF ES+) m/z 813.69 ([M+H]+,100%); HRMS (TOF ES+) calcd for C₄₇H₉₃N₂O₆P[M+H]⁺: 813.6865, found 813.6850.

### Synthesis of Compound 7

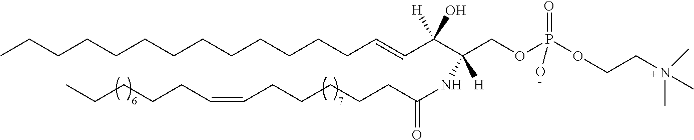

Prepared following general procedure 4 using lyso-sphingomyelin (20 mg, 0.04 mmol) and intermediate 17 (38 mg, 0.08 mmol, 2 eq). The title compound was obtained as an off-white powder after purification (19 mg, 58%). MS (TOF ES+) m/z 813.69 ([M+H]+,100%); HRMS (TOF ES+) calcd for C₄₇H₉₃N₂O₆P[M+H]⁺: 813.6861, found 813.6850.

### Synthesis of Compound 8

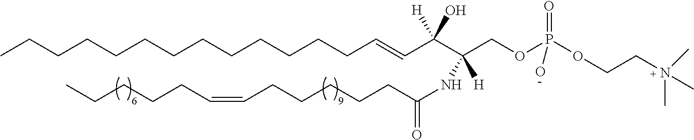

Prepared following general procedure 4 using lyso-sphingomyelin (20 mg, 0.04 mmol) and intermediate 18 (40 mg, 0.08 mmol, 2 eq). The title compound was obtained as an off-white powder after purification (21 mg, 62%). MS (TOF ES+) m/z 841.71 ([M+H]+,100%); HRMS (TOF ES+) calcd for C₄₉H₉₇N₂O₆P[M+H]⁺: 841.7184, found 841.7188.

### Synthesis of Compound 9

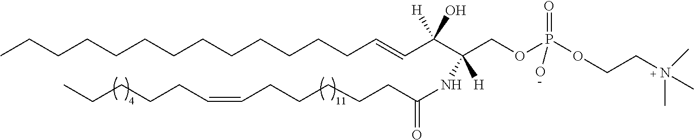

Prepared following general procedure 4 using lyso-sphingomyelin (20 mg, 0.04 mmol) and intermediate 19 (41 mg, 0.08 mmol, 2 eq). The title compound was obtained as an off-white powder after purification (18 mg, 54%). MS (TOF ES+) m/z 841.71 ([M+H]+,100%); HRMS (TOF ES+) calcd for C₄₉H₉₇N₂O₆P[M+H]⁺: 841.7182, found 841.7177.

### Synthesis of Compound 10

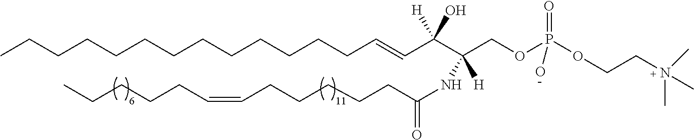

Prepared following general procedure 4 using lyso-sphingomyelin (20 mg, 0.04 mmol) and intermediate 20 (42 mg, 0.08 mmol, 2 eq). The title compound was obtained as an off-white powder after purification (21 mg, 60%). MS (TOF ES+) m/z 869.7 ([M+H]+, 100%); HRMS (TOF ES+) calcd for C₅₁H₁₀₁N₂O₆P[M+H]⁺: 869.7476, found 869.7479.

### Example 2—In Vitro Testing of Sphingomyelin Blocker

Previous work (J. Exp. Med., 2021, 218, 7, e20202699) showed that natural sphingomyelin with a long C42 chain length and two unsaturations (42:2 SM):

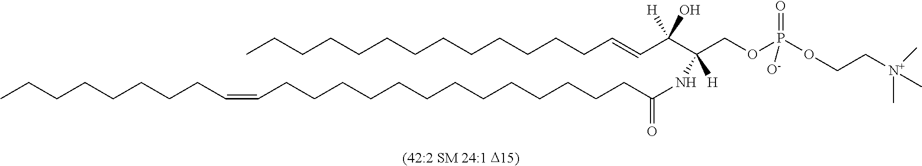 [SMILES: CCCCCCCC/C=C\CCCCCCCCCCCCCC(=O)N[C@@H](COP(=O)([O-])OCC[N+](C)(C)C)[C@H](O)/C=C/CCCCCCCCCCCCC]

blocked CD1a-T cell receptor binding as measured in vitro by CD1a tetramer binding to human T cells with a defined T cell receptor. In sharp contrast, a shorter chain 36:2 SM:

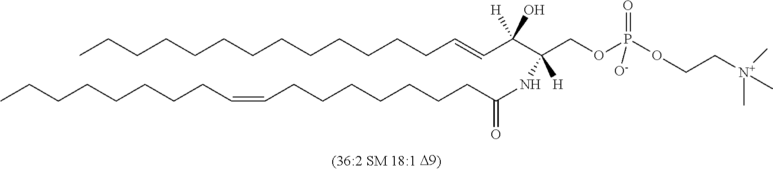 [SMILES: CCCCCCCC/C=C\CCCCCCCC(=O)N[C@@H](COP(=O)([O-])OCC[N+](C)(C)C)[C@H](O)/C=C/CCCCCCCCCCCCC]

did not block binding. In addition, the doubly unsaturated C42:2 compound blocked better than the singly unsaturated C42:1 compound.

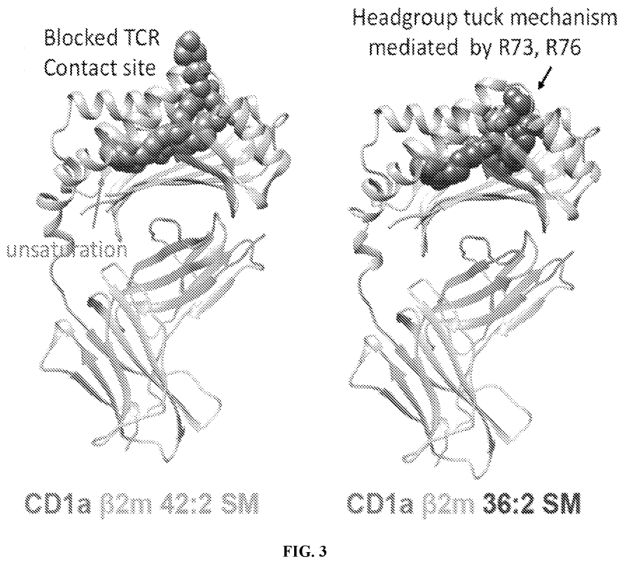

Without being bound by any particular theory or speculation, it is considered that the longer C42:2 SM lipid allows the phosphocholine head group of the lipid to protrude broadly from CD1a to block approaching T cell receptors (See FIGS. 2 and 3). The second unsaturation in the fatty acyl unit is buried deep within the CD1a pocket, where it serves to kink the acyl chain so that it curves to bind more efficiently within the pocket leading to higher potency action (FIG. 3, arrow pointing to unsaturation).

Generally, naturally occurring cellular sphingomyelins within mammalian cells occur as mixtures, and they are most commonly C34-C36 but can range up to C42 in length. However, the sphingomyelins preferentially eluted from CD1a trended strongly towards the rare C42 sphingomyelins with two unsaturations, indicating that chain length >C36 and unsaturation >1 can increase CD1a binding efficiency. The data provided in this example advantageously and unexpectedly showed that synthetic sphingomyelins with particularly long chain length have better binding to CD1a in cells as compared to mixtures of diverse sphingomyelins with diverse chain lengths or defined molecules that recapitulate the most commonly occurring natural chain lengths in the range of C34-C36. As shown in FIG. 3, longer chain lipids bind within CD1a, such that the longer lipids for the choline head group to more extensively protruding from within CD1a, which blocks T cell receptor approach and binding to CD1a. Therefore, these long chain non-natural compounds were shown to be effective blockers of CD1a binding to TCRs in CD1a tetramer assays using human CD1a-reactive T cells based on the effects of their long chain length to generate ligand protrusion. The synthetic sphingomyelin ligands are provided in Table 1 above.

The exemplified compounds were prepared in accordance with the synthetic scheme shown in Scheme 1. Chain length, position of unsaturation, chemical formula, and exact mass for exemplified synthetic sphingomyelins are summarized in Table 2.

TABLE 2

| | Double bond | Chemical formula |
|---|---|---|
| Cmpd. No. | position | Exact mass |
| 1 | 38:2 SMΔ9 | C43H85N2O6P |
| | (20:1 Δ9) | 756.6145 |
| 2 | 38:2 SMΔ11 | C43H85N2O6P |
| | (20:1 Δ11) | 756.6145 |
| 3 | 40:2 SMΔ9 | C45H89N2O6P |
| | (22:1 Δ9) | 784.6458 |
| 4 | 40:2 SMΔ11 | C45H89N2O6P |
| | (22:1 Δ11) | 784.6458 |
| 5 | 40:2 SMΔ13 | C45H89N2O6P |
| | (22:1 Δ13) | 784.6458 |
| 6 | 42:2 SMΔ11 | C47H93N2O6P |
| | (24:1 Δ11) | 812.6771 |
| 7 | 42:2 SMΔ13 | C47H93N2O6P |
| | (24:1 Δ13) | 812.6771 |
| 8 | 44:2 SMΔ15 | C49H97N2O6P |
| | (26:1 Δ15) | 840.7084 |
| 9 | 44:2 SMΔ17 | C49H97N2O6P |
| | (26:1 Δ17) | 840.7084 |
| 10 | 46:2 SMΔ17 | C51H101N2O6P |
| | (28:1 Δ17) | 868.7397 |

All compounds contain C18:1 sphingosine coupled to monounsaturated fatty acyl units ranging from C20 to C28, so that the overall combined length of the sphingosine and fatty acyl chains ranged from C38 to C46, so that they are longer than the most common natural sphingomyelins (C34-C36). The fatty acyl units were synthesized from defined precursors that allowed unsaturation to be placed at differing positions in the acyl chain from C9 (Δ9) to C17 (Δ17) (See Scheme 1). As the data showed, the position of double bond in the fatty acid affected CD1a binding, since it is inserted inside the CD1a cleft (FIG. 2).

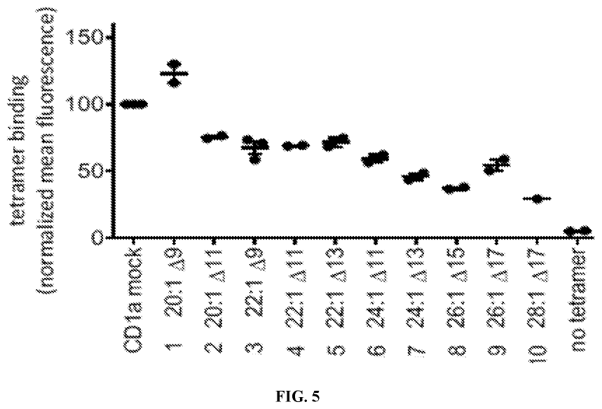

The degree of inhibition for CD1a protein binding to T cells expressing receptors that normally bind to CD1a was tested for the exemplified compounds 1-10 (see FIG. 5). Increased chain length contributed to blockade in all cases, and the non-natural positioning of the second unsaturation in the fatty acyl unit affected blockade. More blockade was seen when the unsaturation was generated in positions more distal to the head group located at positions 15-17 (Δ15, Δ17). The strongest blockade among these newly synthesized compounds was with the longest lipid, C46:2 SM 417 (also known as compound 10), which is longer than most or all naturally occurring sphingomyelins. Also the somewhat improved blocking of C44Δ15 SM as compared to C44Δ17 SM suggests that efficacy of compound 10 (C46Δ17 SM) can be further improved by moving the unsaturation more proximally to create C46Δ15 SM. Accordingly, additional SM blockers include those with increased length of alkyl chains beyond C46, where the unsaturation is moved to more proximal positions in the fatty acyl chain and the number of unsaturations in the fatty acid chain is increased. Also, the compounds contain terminal or near terminal alkane branches (isomethyls or anteisomethyls) in the fatty acyl chains.

In contrast, naturally occurring mixtures of endogenous lipids in CD1a (FIG. 5, ‘mock’) with varied chain length and head groups do not block T cell binding. Also, the commonly occurring short chain sphingomyelin ligands in the range of C34-38 had little blocking or slight augmentation of CD1a binding to T cells (FIG. 5). Thus, the data showed that long chain length is an essential component of the blocking response and the position of the unsaturation affects response. Also, mixed sphingomyelins or mixed lipids, as found in natural lipid preparations known as CD1-endogenous lipids (CD1-endo), cause activation rather than blockade of T cells in published studies.

Overall, the natural cellular lipids are not suitable for use as T cell inhibitory drugs because they are present in trace amounts (nanograms) in cells, contain mixtures of short chain T cell agonists and long chain T cell antagonists, and do not represent the full spectrum of the kinds of synthetic lipids that would bind CD1 and T cell receptors. In contrast, pure lipids synthesized with long chain length and headgroup distal unsaturations strongly blocked CD1a binding to T cell receptors, as well as inflammation, and terminal methyl branching in fatty acids can influence lipid binding to CD1a, so blockers can contain distal alkyl branching patterns.

The most advanced prototype of a CD1 blocker compound is a synthetic lipid designated here as C46:2 Δ17 sphingomyelin (compound 10). As the experimental data in this example shows, this compound strongly blocked CD1a binding to human T cell receptors in vitro. As shown in the following Example 2, formulated in ethanol and applied to human CD1a expressing mice, compound 10 reduced skin swelling and cellular inflammation in vivo in a model of psoriasiform inflammation. Strong inflammation was seen only in CD1a transgenic mice, and the structure-function analysis shows a correlation between CD1a-T cell receptor blockade in vitro with human T cells and therapeutic response in the skin. Thus, the data support that the mechanism of action occurs specifically via CD1a and T cell response, suggesting that unwanted side effects seen with steroids and other broadly acting T cell reagents might be lacking in CD1a blocking lipids.

### Example 3—Formulation of Sphingomyelin Blockers for Topical Administration

CD1a is normally expressed on Langerhans cells in the epidermal layer of human skin. Because mice do not express CD1a, the in vivo experiments were conducted with a human CD1a knock-in mouse. CD1a is non-polymorphic, and about 1 percent of human skin T cells stain with CD1a tetramers suggesting the presence of large skin resident populations in vivo. The lipids of this disclosure block CD1a binding to several T cell clones in vitro and also more broadly block the response of larger populations of polyclonal T cells that recognize CD1a in vivo, leading to decreased inflammation in the skin.

Synthetic long (C46:2 SMΔ17, compound 10), intermediate (C42:2Δ15 SM) and short chain (C34:1 SM) sphingomyelins were compared for inflammatory response. Unlike natural preparations that contain mixtures of short and long chain lipids, these experiments used pure synthetic lipids to titrate CD1a binding and inhibition of immune response. The functional requirements for the delivery vehicle are that it must: 1) dissolve amphipathic lipids, 2) be safe, immunologically unreactive, and non-toxic to skin and 3) create a lipid film that can subsequently penetrate to the epidermal layers, where CD1a is normally located in human skin and in the skin of CD1a transgenic mice. In this experiment, ethanol was chosen which is a chemically simple, safe and effective vehicle; however, other vehicles can also be used.

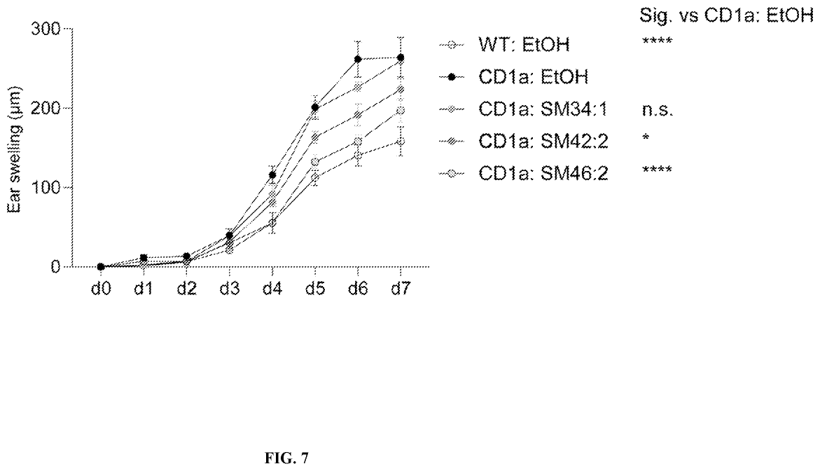

A widely used model of psoriasiform and dermatitic inflammation induced by Aldara cream, whose active ingredient is a toll-like receptor 7 agonist (imiquimod), was used in this example. Experiments were carried out in which lipid antagonists were applied to the skin of human CD1a transgenic mice in an ethanol carrier, which dried rapidly, leaving a lipid film on the skin. A significant reduction in ear swelling was observed, as a global measure of inflammation and psoriasiform and dermatitic disease when long chain sphingomyelins were applied before (data not shown) or simultaneously with Aldara treatment (FIGS. 7-11).

Two controls indicate that the immunological effects are mediated by CD1a, rather than some general emollient effect of lipid application to the skin. First, no immune response was seen in wild type mice lacking CD1a. Second, the negative control, C34:1 SM, which is not a T cell receptor blocker in tetramer assays, but serves a negative control for skin softening or other non-specific effects of sphingomyelin lipids, showed no statistically significant effects on ear thickening and fewer effects in the measurements of cellular and cytokine response.

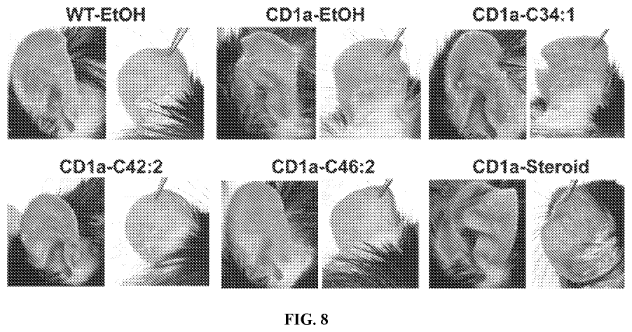

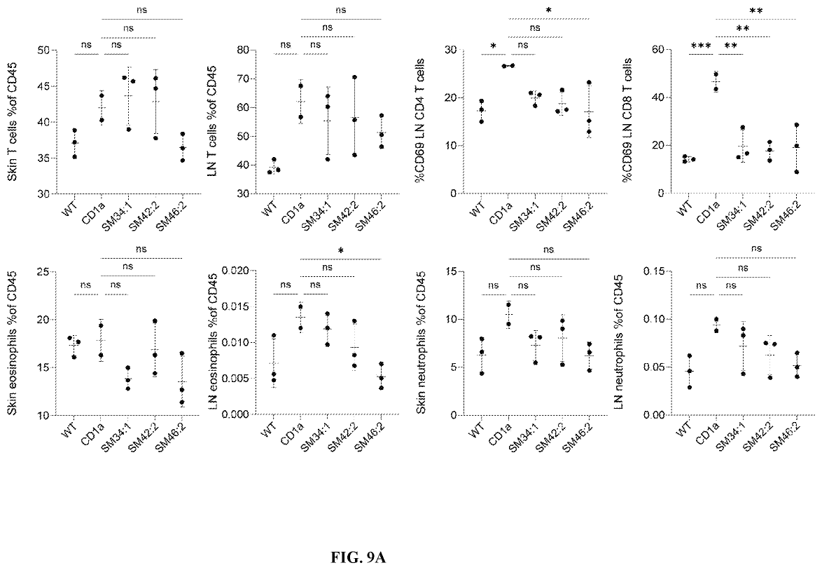

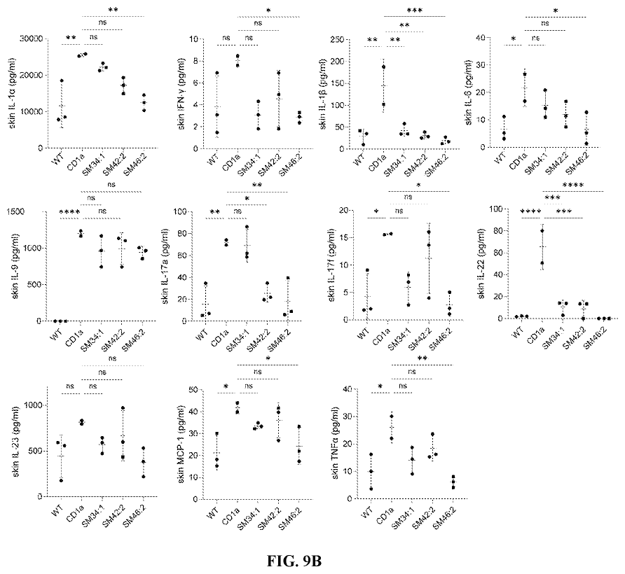

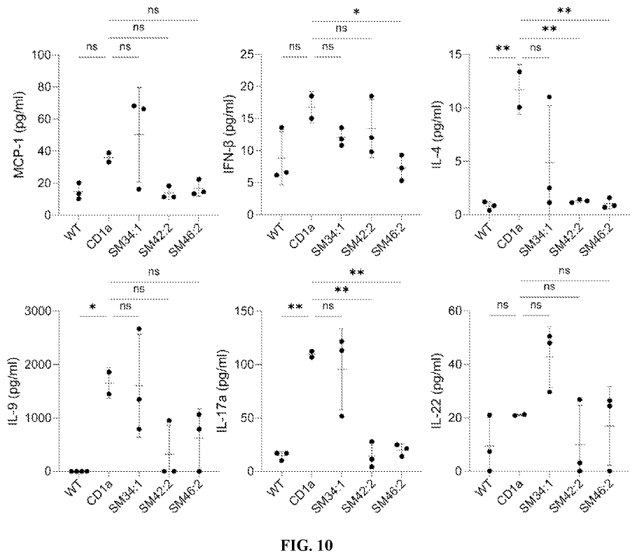

Images of treated skin (FIG. 8) confirmed the patterns seen in ear thickness measurements and detection of cellular inflammation in the skin. Reduced inflammatory lesions were observed in skin treated with C42:2 SM and C46:2 SM as compared to short chain C34:1 SM (FIG. 8) with the strongest effects seen with C46:2, which showed significantly reduced skin inflammation. High numbers of resident lymphocytes normally present in uninflammed skin so, as expected, treatments did not broadly reduce key resident cell populations, but there were some reductions in total T cell, neutrophil and eosinophil counts in ear skin and draining lymph node (FIG. 9A). Instead, the in vitro experimental data point to a likely reduction in T cell activation (rather than total numbers). Consistent with this expectation, surface markers of T cell activation (CD69) and skin cytokines that are normally released during activation, including IL1α, IFN-γ, IL-1β, IL-6, IL17α, IL-17f, TNFα and most strikingly, IL-22, showed significant decreases in mice treated with compound SM46:2 (FIG. 9B). Also, although the immune stimulus was initiated in the skin, systemic measures of immune response, including plasma cytokines IFNβ, IL-4, IL17α, (FIG. 10) were significantly reduced by C46:2 SM. In addition, other cytokine reductions were apparent in mice treated with synthetic SMs that did not reach statistical significance in these experiments with small group numbers. These trend changes were consistent with broad reduction in skin and systemic inflammation.

Overall, the small but detectable cellular changes were supported by data showing a strong and wide impact on skin and plasma cytokine pathways including members of type 1, type 2 and type 17/22 families with relevance for many human skin inflammatory diseases and systemic associations (FIG. 10). In particular, IFNγ, IL-4, IL-17, IL-22, IL1β and TNFα have proven roles in T cell mediated skin disease or have been successfully targeted with biological therapies in human skin disease. Perhaps the most useful general and objective indicator of inflammation is overall ear thickening (FIG. 7) and visible skin change (FIG. 8), which were most significantly inhibited by compound 10, as compared to SM preparations with shorter lipid tails.

Inhibition of CD1a-mediated T cell response is not expected to hinder all forms of immune response. The data show that some cell types (FIG. 8), local cytokines (FIG. 9) and systemic responses (FIG. 10) are affected more than others. The statistically significant effects of compound 10, including generally stronger effects than observed with shorter chain SMs, were seen for activation markers on T cells, as well T cell derived cytokines, IL-17a, IL-17f, IL-22, TNF-α and IL-4. These effector functions have been previously associated with CD1a-autoreactive T cells in human skin diseases, including atopic dermatitis and psoriasis. Thus, particular immune outcomes observed are consistent with specific effects mediated via inhibition of CD1a autoreactive rather than global immune inhibition, and they are specifically tied to the non-natural C46:2 structure of the synthetic lipid analog.

These results demonstrated efficacy of a simple ethanol-based treatment formulation providing credible evidence for broader efficacy of creams and ointments to promote penetration of the exemplified compounds to the epidermis, where CD1a is localized.

A large fraction of human skin T cells are CD1a autoreactive, and they are activated in psoriasis, atopic dermatitis and contact dermatitis. But there are currently no therapeutic approaches that directly target CD1a binding lipid ligands. The present examples identify the molecules, molecular mechanisms and efficacy of non-natural synthetic CD1a ligands in selective inhibition of CD1a-dependent T cell responses in vitro in humans and in vivo in humanized mice. The compounds of the present examples represent a fundamentally new therapeutic approach based on selective inhibition of CD1-mediated T cell responses as distinguished from MHC-peptide and other T cell responses. The experimental data provide credible evidence that the compounds are useful to treat human diseases, e.g., in which CD1a has been implicated, including 1) T cell responses to sebaceous oils (squalene) that are present in common acne and acne vulgaris lesions, 2) contact dermatitis from skin creams, balsam of Peru, farnesol, poison ivy, sulfa drugs, and other contact allergens, 3) psoriatic inflammation, 4) systemic type IV hypersensitivity responses to bee, wasp, and dust mite allergens and 5) chronic atopic dermatitis. These are all major diseases for which current therapy is suboptimal or for which current therapies generate unwanted, off-target effects based on their broader mechanisms of action. The 46:2 SM (compound 10) has been produced synthetically in milligram quantities, and the basic ethanol application system has been validated to show protection from disease in an in vivo mouse model.

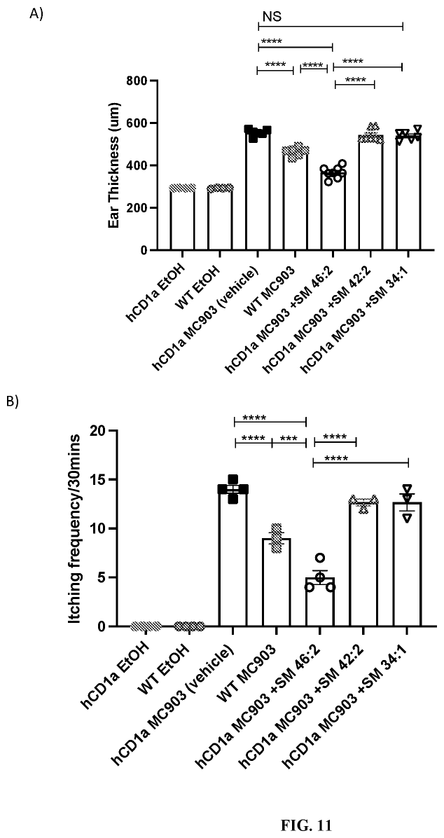

In order to investigate whether SM had efficacy in a different inflammatory skin model, we utilized the MC903-induced model of type 2 cytokine inflammation which has features of dermatitis in humans and prominently causes itch, a major treatable skin symptom. The MC903-induced type 2 inflammation is amplified in the presence of hCD1a (Hardman et al Nat Comms 2022). SM46:2 significantly reduced ear thickness and itching/pruritus scores below the vehicle control in the MC903-induced skin inflammatory response. Furthermore, an additional discovery was made that the SM46:2 significantly reduced ear thickness and pruritus below the wild-type mice treated with MC903 (FIG. 11). These mice normally express CD1d and then were engineered to express CD1a. The suppression of response in wild type non-transgenic mice suggests that the SM46:2 has capacity to act as a dual blocker of hCD1a and mCD1d, and we have detected long chain sphingomyelins in association with CD1d proteins in cells (Huang, Cell, 2023, in presss). As well as having potential therapeutic applications in diseases involving type 2 inflammation in humans, the data support utility in treating broader diseases where pruritus and itch are symptoms. CD1a has not been previously linked with pruritus and so this represents a new area of biology and opens new areas for therapeutic application.

### Prophetic Example 4—Sulfatides

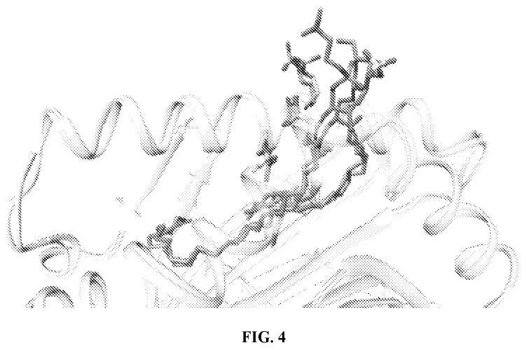

Sulfatides (compounds S1-S10) have the same kinds of CD1a binding lipid tails as the synthetic sphingomyelins, but carry a differing headgroup comprised of sulfate and carbohydrate rather than phosphocholine. After in vitro evidence showed that sphingomyelins can block human CD1a-reactive T cell response, CD1a was co-crystallized with sulfatide (FIG. 4), showing a molecular mechanism that can account for T cell blockade that is related to but somewhat different then the stearic interference mechanism for sphingomyelins (FIG. 3). In both cases CD1a binding is mediated by alkyl chain interactions inside of the CD1a cleft, suggesting that the length and saturation patterns observed for SMs with apply here. However, the head group of sulfatide has two proposed mechanisms for blocking TCR contact. Overlay of CD1a-sulfatide shows that the large sulfate containing moiety occupied a similar space compared to the choline group of sphingomyelin, which is the blocking element for SM. This positioning suggests that the sulfo-sugar could sterically inhibit TCR approach in a manner analogous to the choline group in sphingomyelin (FIGS. 2 and 4, see Example 1). However, the sulfatide moiety also disrupts a triad of residues on the outer surface of CD1a, which changes the intrinsic conformation of CD1a at the site at which the TCR can bind. These structural data predict that sulfatides could be particularly broad and efficacious blockers of T cell response.

The synthesis of sulphated analogues of glycosyl ceramides S1-S10 follows the synthetic route outlined above for sphingomyelins utilizing the corresponding fatty acids generated during the chemical synthesis of the compounds above (Scheme 1).

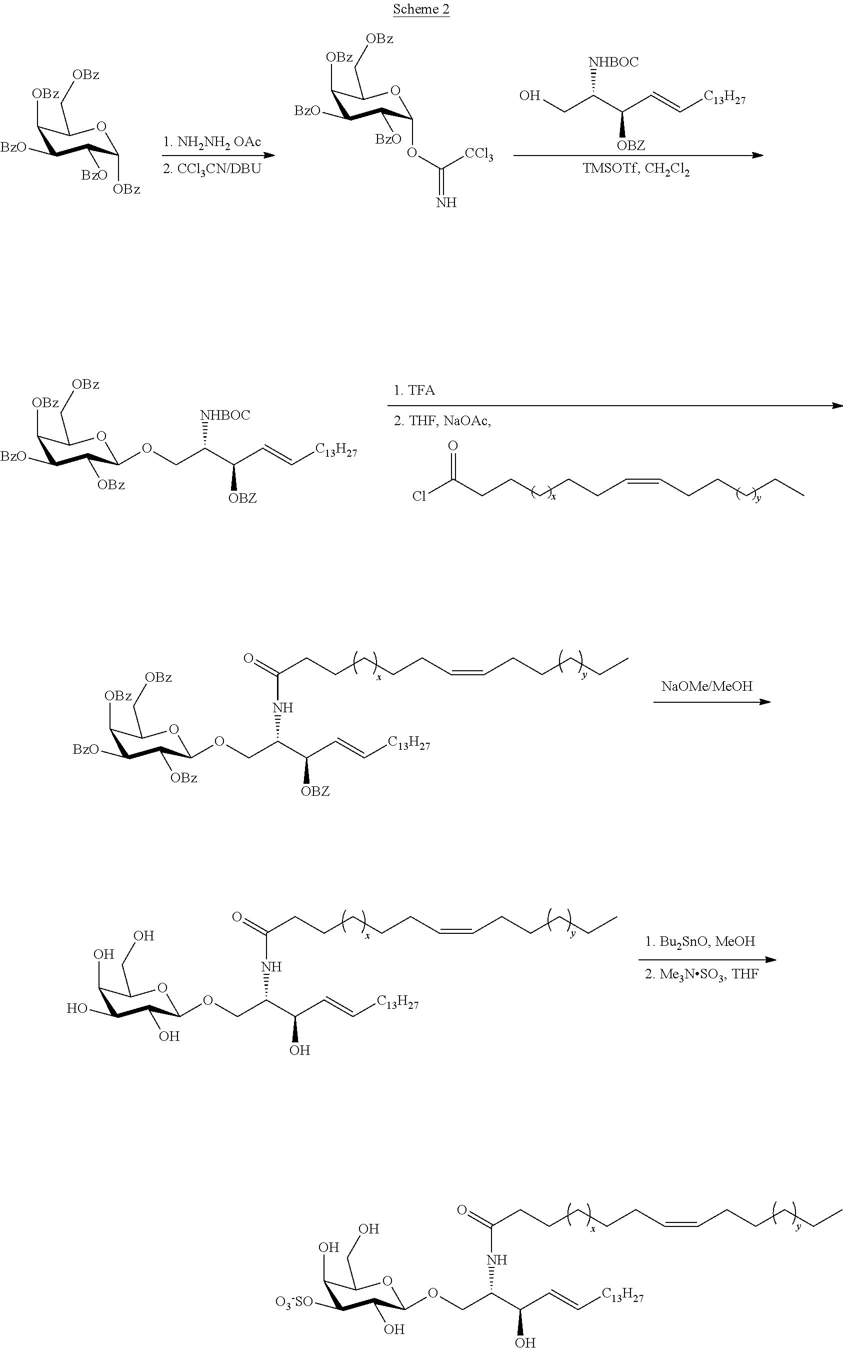

The sulfatide headgroup is chemically grafted onto the length and saturation optimized sphingolipid tails described in example 3. The following sulfatide compounds shown in Table S1 can be prepared according to analogous methods and procedures shown for sphingomyelins in Scheme 2 above.

TABLE S1

| Com- | |
|---|---|
| pound | |
| No. | Chemical structure |
| S1 | 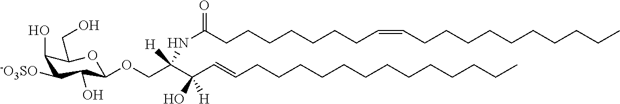 |
| S2 | 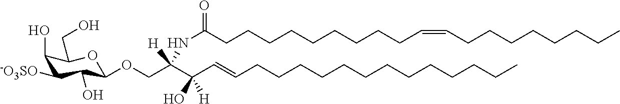 |
| S3 | 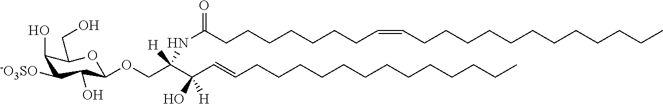 |
| S4 | 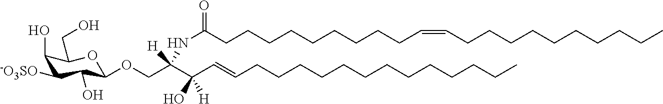 |
| S5 | 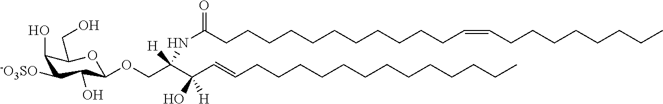 |
| S6 | 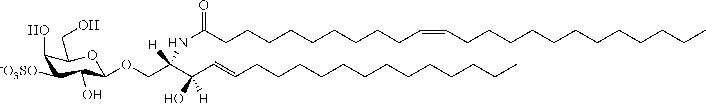 |
| S7 | 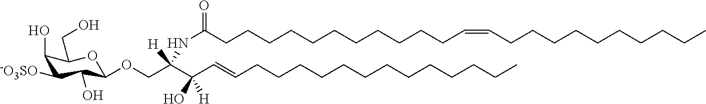 |
| S8 | 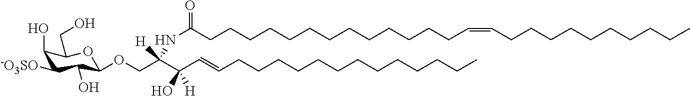 |
| S9 | 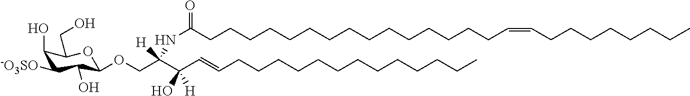 |
| S10 | 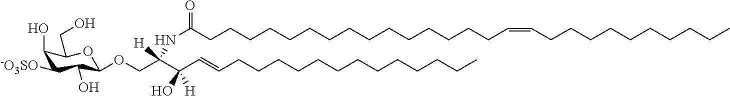 |

Chain length, position of unsaturation, chemical formula, and exact mass for exemplified synthetic sulfatides are summarized in Table S2.

TABLE S2

| | Double bond | Chemical formula |
|---|---|---|
| Cmpd. No. | position | Exact mass |
| S1 | 38:2 Δ9 | Chemical Formula: C44H82NO11S- |
| | (20:1 Δ9) | Exact Mass: 832.5614 |
| S2 | 38:2 Δ11 | Chemical Formula: C44H82NO11S- |
| | (20:1 Δ11) | Exact Mass: 832.5614 |
| S3 | 40:2 Δ9 | Chemical Formula: C46H86NO11S- |
| | (22:1 Δ9) | Exact Mass: 860.5927 |
| S4 | 40:2 Δ11 | Chemical Formula: C46H86NO11S- |
| | (22:1 Δ11) | Exact Mass: 860.5927 |
| S5 | 40:2 Δ13 | Chemical Formula: C46H86NO11S- |
| | (22:1 Δ13) | Exact Mass: 860.5927 |
| S6 | 42:2 Δ11 | Chemical Formula: C48H90NO11S- |
| | (24:1 Δ11) | Exact Mass: 888.6240 |
| S7 | 42:2 Δ13 | Chemical Formula: C48H90NO11S- |
| | (24:1 Δ13) | Exact Mass: 888.6240 |
| S8 | 44:2 Δ15 | Chemical Formula: C50H94NO11S- |
| | (26:1 Δ15) | Exact Mass: 916.6553 |
| S9 | 44:2 Δ17 | Chemical Formula: C50H94NO11S- |
| | (26:1 Δ17) | Exact Mass: 916.6553 |
| S10 | 46:2 Δ17 | Chemical Formula: C52H98NO11S- |
| | (28:1 Δ17) | Exact Mass: 944.6866 |

1. A compound of Formula (I): 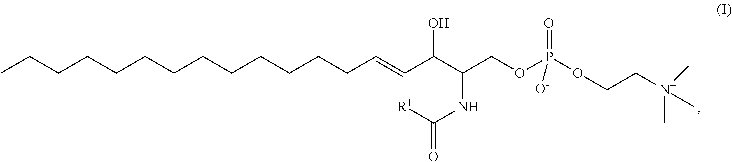
or a pharmaceutically acceptable salt thereof, wherein R¹ is C₂₇₋₄₁ alkenyl comprising 1 or 2 double bonds.

2. The compound of claim 1, wherein R¹ comprises 2 double bonds.

3. The compound of claim 1, wherein R¹ comprises 1 double bond.

4. The compound of claim 3, wherein the double bond is at C₉, C₁₁, C₁₃, C₁₅, C₁₇, or C₁₉.

5. The compound of any one of claims 1-4, wherein each double bond is in Z configuration.

6. The compound of any one of claims 1-4, wherein each double bond is in E configuration.

7. The compound of any one of claims 1-6, in which the fatty acid or sphingosine chains have isomethyl or anteisomethyl branches.

8. The compound of any one of claims 1-7, wherein the compound has formula: 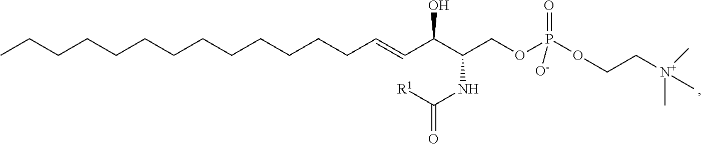
or a pharmaceutically acceptable salt thereof.

9. The compound of claim 1, wherein the compound of Formula (I) has formula: 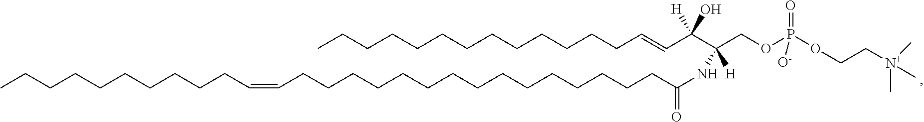 [SMILES: CCCCCCCCCC/C=C\CCCCCCCCCCCCCCCC(=O)N[C@@H](COP(=O)([O-])OCC[N+](C)(C)C)[C@H](O)/C=C/CCCCCCCCCCCCC]
or a pharmaceutically acceptable salt thereof.

10. A compound selected from any one of the following compounds: 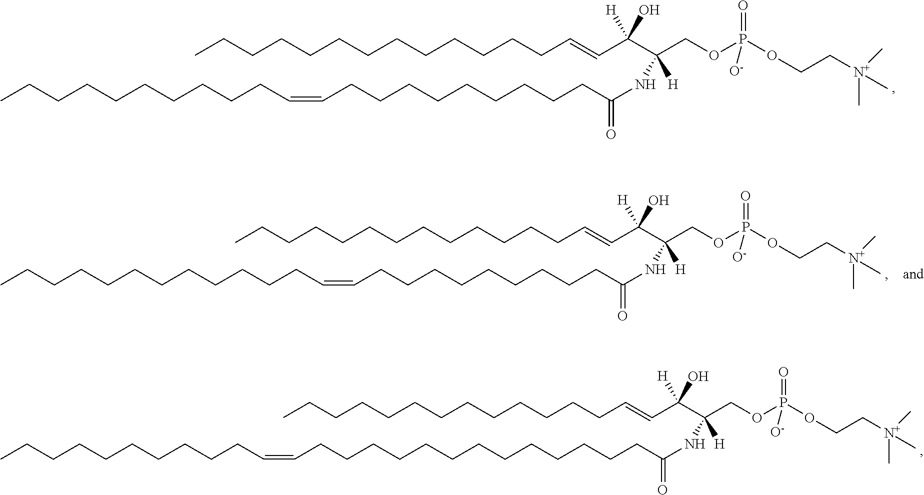
or a pharmaceutically acceptable salt thereof.

11. A pharmaceutical composition comprising a compound of any one of claims 1-10, or a pharmaceutically acceptable salt thereof, and a pharmaceutically acceptable carrier.

12. The pharmaceutical composition of claim 11, which is suitable for enteral, parenteral or topical application to skin or mucosa.

13. The pharmaceutical composition of claim 11, wherein the composition is in a dosage form selected from solution, cream, ointment, lotion, and mousse.

14. A method of preventing or treating an inflammatory disease, the method comprising administering to a subject in need thereof a therapeutically effective amount of a compound of any one of claims 1-10, or a pharmaceutically acceptable salt thereof, or a pharmaceutical composition of any one of claims 11-13.

15. The method of claim 14, wherein the inflammatory disease is selected from diabetes, rheumatoid arthritis, colitis, ulcerative colitis, asthma, allergic asthma, fatty liver disease, obesity, insulin resistance, inflammatory diseases of the liver and gut, steatohepatitis, liver inflammation, acne, autoimmune diseases, atherosclerosis, hyperlipidemia, dyslipidemia, rhinitis, conjunctivitis, oral inflammation, thyroid disease, lung disease, inflammatory bowel disease (IBD), Crohn's disease, and chronic obstructive airways disease.

16. The method of claim 14, wherein the inflammatory disease is an inflammatory skin disease.

17. The method of claim 16, wherein the inflammatory skin disease is selected from pruritus, nodular prurigo, itch, atopic dermatitis, eczema, psoriasis, common acne, acne vulgaris, urticaria, seborrheic dermatitis, nummular dermatitis, dermatitis herpetiformis, poison ivy (urushiol) dermatitis, Balsam of Peru dermatitis, contact dermatitis of known or unknown origin, lupus erythematosus, drug reactions (cutaneous and systemic, including to sulfa antibiotics), Stevens-Johnson syndrome, toxic epidermal necrolysis, graft vs host disease, vasculitis, palmoplantar pustulosis, scleroderma, systemic sclerosis, autoimmune blistering conditions, vitiligo, alopecia areata, alopecia universalis, alopecia totalis, lichen planus, erythema multiforme, urticaria, other pruritic conditions, and other allergic and irritant skin diseases.

18. A compound of Formula (II): 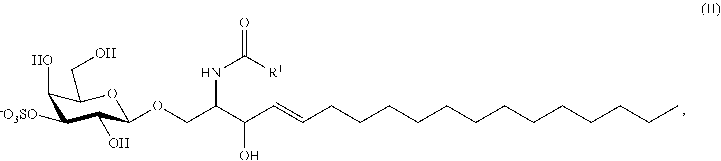
or a pharmaceutically acceptable salt thereof, wherein R¹ is C₂₇₋₄₁ alkenyl comprising 1 or 2 double bonds.

19. The compound of claim 18, wherein R¹ comprises 2 double bonds.

20. The compound of claim 18, wherein R¹ comprises 1 double bond.

21. The compound of claim 20, wherein the double bond is at C9, C11, C13, C15, C17, or C19.

22. The compound of any one of claims 18-21, wherein each double bond is in Z configuration.

23. The compound of any one of claims 18-21, wherein each double bond is in E configuration.

24. The compound of any one of claims 18-23 in which the fatty acid or sphingosine chains have isomethyl or anteisomethyl branches.

25. The compound of any one of claims 18-23, wherein the compound has formula: 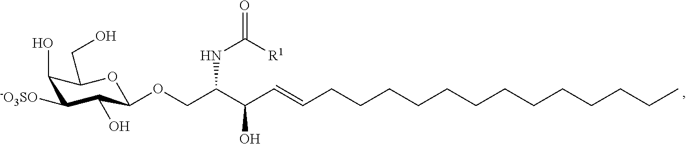
or a pharmaceutically acceptable salt thereof.

26. The compound of claim 18, wherein the compound of Formula (II) has formula: 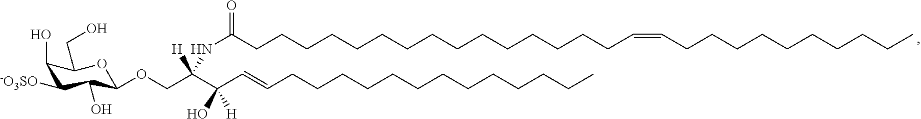
or a pharmaceutically acceptable salt thereof.

27. A compound selected from any one of the following compounds: 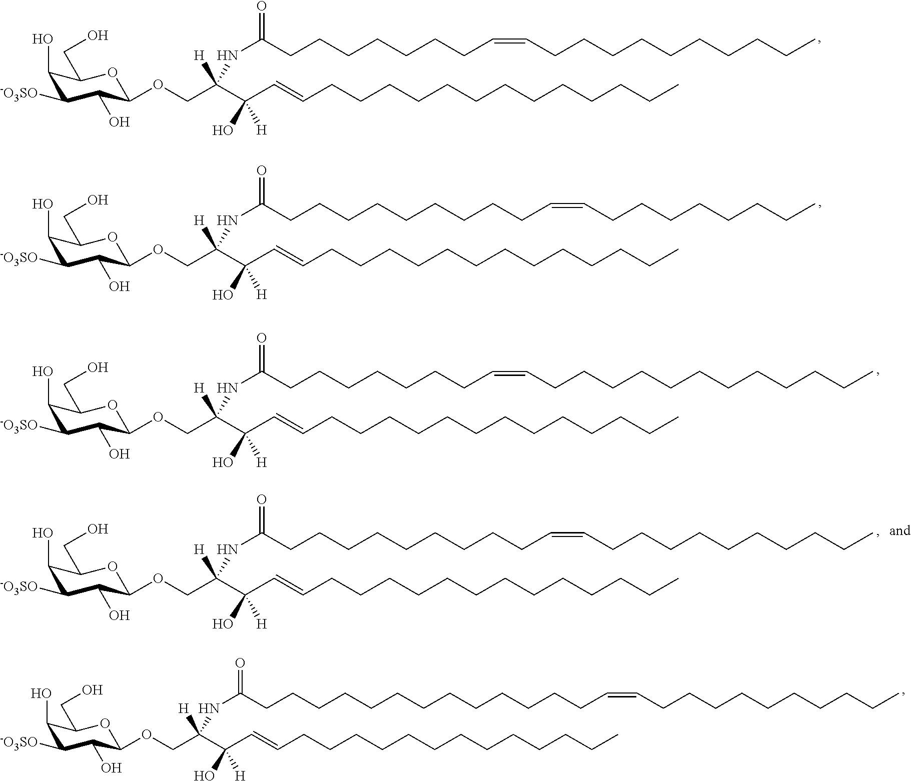
or a pharmaceutically acceptable salt of any of the foregoing.

28. A pharmaceutical composition comprising a compound of any one of claims 18-27, or a pharmaceutically acceptable salt thereof, and a pharmaceutically acceptable carrier.

29. The pharmaceutical composition of claim 28, which is suitable for enteral, parenteral or topical application to skin or mucosa.

30. The pharmaceutical composition of claim 28, wherein the composition is in a dosage form selected from solution, cream, ointment, lotion, and mousse.

31. A method of preventing or treating an inflammatory disease, the method comprising administering to a subject in need thereof a therapeutically effective amount of a compound of any one of claims 18-27, or a pharmaceutically acceptable salt thereof, or a pharmaceutical composition of any one of claims 28-30.

32. The method of claim 31, wherein the inflammatory disease is selected from diabetes, rheumatoid arthritis, colitis, ulcerative colitis, asthma, allergic asthma, fatty liver disease, obesity, insulin resistance, inflammatory diseases of the liver and gut, steatohepatitis, liver inflammation, acne, autoimmune diseases, atherosclerosis, hyperlipidemia, dyslipidemia, rhinitis, conjunctivitis, oral inflammation, thyroid disease, lung disease, inflammatory bowel disease (IBD), Crohn's disease, and chronic obstructive airways disease.

33. The method of claim 31, wherein the inflammatory disease is an inflammatory skin disease.

34. The method of claim 33, wherein the inflammatory skin disease is selected from atopic dermatitis, eczema, psoriasis, common acne, acne vulgaris, urticaria, seborrheic dermatitis, nummular dermatitis, dermatitis herpetiformis, poison ivy (urushiol) dermatitis, Balsam of Peru dermatitis, contact dermatitis of known or unknown origin, lupus erythematosus, drug reactions (cutaneous and systemic, including to sulfa antibiotics), Stevens-Johnson syndrome, toxic epidermal necrolysis, graft vs host disease, vasculitis, palmoplantar pustulosis, scleroderma, systemic sclerosis, autoimmune blistering conditions, vitiligo, alopecia areata, alopecia universalis, alopecia totalis, lichen planus, erythema multiforme, urticaria, and other pruritic conditions, and other allergic and irritant skin diseases.
